(19) United States
(12) Patent Application Publication
(10) Pub. No.: US 2019/0365305 A1
(43) Pub. Date: Dec. 5, 2019

(54) SYSTEMS AND METHODS FOR PERFORMING DIGITAL PSYCHOMETRY

(71) Applicant: DIGI-PSYCH INC., Albany, NY (US)

(72) Inventors: Foroud Hamidian, Tehran (IR); Mona Fazeli, Tehran (IR)

(21) Appl. No.: 15/992,705

(22) Filed: May 30, 2018

(51) Int. Cl. A61B 5/16; G10L 15/08; G10L 25/63; G10L 25/66; G06F 17/27; A61B 5/00

(52) U.S. Cl. CPC A61B 5/16; G10L 15/08; G10L 25/63; G10L 25/66; A61B 5/4088; A61B 5/165; A61B 5/167; A61B 5/168; G06F 17/2785

(57) ABSTRACT

Methods and systems for analyzing one or more psychological states of a user are provided. The method includes analyzing a plurality of words and organizing each word in the plurality of words into one or more categories based on the analysis of the plurality of words. The method further includes receiving a plurality of words from the user, analyzing the plurality of words from the user, determining, which category or categories to which one or more words in the plurality of words from the user belongs, determining, based on the analysis of the plurality of words from the user and the determination of which category or categories to which the one or more words in the plurality of words from the user belongs, one or more psychological states of the user, and outputting, to a graphical user interface, the one or more psychological states.

### CLAIM OF PRIORITY

This application is a United States non-provisional application and claims no priority to any previous United States patent application.

### FIELD OF THE EMBODIMENTS

This invention relates to speech analysis and, in particular, to systems and methods for analyzing speech to determine one or more psychological traits.

### BACKGROUND OF THE EMBODIMENTS

There is a great deal of information that can be extrapolated from analyzing a person's speech and writing style. This includes not just the information related to the subject matter of the speech, but also from the nuances and word choices the user makes, enabling a greater understanding of the user. However, up to this point, it has been difficult to easily and efficiently analyze speech to adequately extrapolate information about the speaker.

For at least these reasons, an easy and efficient means of analyzing the speech of one or more users to determine various psychological traits and perform various psychological assessments of the one or more users is needed.

Examples of related art are described below:

U.S. Pat. No. 8,461,148 generally describes the treatment of compulsive, impulsive and pervasive developmental disorders. More particularly, the methods described herein comprise administration of memantine to an individual suffering from such a disorder in an amount effective to relieve one or more symptoms of said disorder. In particularly preferred aspects, the invention is directed to the use of memantine for the treatment of autism.

U.S. Pat. No. 9,358,458 generally describes Optional commitments may be provided to players within a videogame. The optional commitments to commit players to perform and/or abstain from certain actions within a videogame. The players may be rewarded for accepting commitments. The players may be penalized for failing to fulfill commitments. This model for rewarding optional behaviors may provide an alternative for incenting actions within a videogame to conventional models in which players are rewarded and/or penalized only after attempting an activity or activities.

U.S. Pat. No. 9,415,314 generally describes methods in which, to simulate group play, player-controlled characters may be grouped prior to entry into a set of asynchronous videogame content. Then separate instances of the set of asynchronous videogame content may be provided to players associated with individual ones of the grouped player-controlled characters. In the separate instance provided to a given player, secondary instances of the player-controlled characters other than the player-controlled character being controlled by the given player may be controlled automatically to accompany the player-controlled character being controlled by the given player.

U.S. Pat. No. 9,693,724 generally describes a system and method that enables a person to unobtrusively quantify the effect of mobility, physical activity, learning, social interaction and diet on cognitive function. The method records on the electronic device one of global positioning system longitude and latitude coordinates, accelerometer coordinates, and gyroscope coordinates, one of outgoing and incoming phone calls, outgoing and incoming emails, and outgoing and incoming text messages, one of URLs visited on an internet browser application, books read on an e-reader application, games played on game applications, and the nutritional content of food consumed, performs the step of learning a function mapping from those recordings to measurements of cognitive function using a loss function to identify a set of optimal weights that produce a minimum for the loss function, uses those optimal weights to create the function mapping, and performs the step of computing the variance of the cognitive function measurements that is explained by the function mapping to assign an attribution to the effect of physical activity on measured changes in cognitive function.

U.S. Patent Publication No. 2012/0330869 generally describes a mental-model elicitation process and apparatus, called the Mental-Model Elicitation Device (MMED). The MMED is used to give rise to more effective end-user mental-modeling activities that require executive function and working memory functionality. The method and apparatus is visual analysis based, allowing visual and other sensory representations to be given to thoughts, attitudes, and interpretations of a user about a given visualization of a mental-model, or aggregations of such visualizations and their respective blending.

U.S. Patent Publication No. 2014/0006042 generally describes methods, devices, systems, and computer readable medium for improving studies such as clinical trials and for improving clinical practice. The methods, devices, systems, and computer readable medium provided herein can be used to identify outlier data in a study, select data collection sites likely to produce high quality data, detect fraud, identify placebo responders, and/or identify likely responders to a therapy. The methods, devices, systems, and computer readable medium provided herein can also be used to optimize a test, for example, a neurocognitive battery, for maximum sensitivity.

U.S. Patent Publication No. 2014/0278786 generally describes a perceptual evaluation system and method that collects, categorizes, evaluates, connects and manages the primary market research of items and combines it with secondary market research to create perception profiles of people and their consumption behaviors, and generates recommendations based on people's perceptions. The system and method enable the perception evaluation of items by users via (click or touch) drag-and-drop mechanisms in a survey processes and applies a novel perception rating scale to categorize, evaluate and cohere the items. The items may include word text, audiovisuals and maps, and the system is deployable on Web and mobile browsers. Web and mobile devices and smart TV devices. The system's output includes recommendations presented as perception maps and other data visualizations.

U.S. Patent Publication No. 2015/0269244 generally describes methods in which, during an analysis technique, organization data for an organization (such as a company) and a set of potential predictors for retention are analyzed to generate Kaplau-Meier estimator curves. Then, clustering analysis is performed to determine natural groupings of Kaplan-Meier estimator curves. Note that the retention data may include, as a function of time, retention probabilities that the individuals remain in functions in an organization and a set of potential predictors for the retention probabilities. Moreover, the predictors for retention in the set of potential predictors are identified based on the determined natural groupings. For example, the identified predictors may be those for which at least two natural groupings have a large centroid separation. Furthermore, the identified predictors for retention may be used to determine remedial action to increase the retention probabilities.

None of the art described above addresses all of the issues that the present invention does.

### SUMMARY OF THE EMBODIMENTS

According to an aspect of the present invention, a method for analyzing one or more psychological states of a user is provided. The method includes analyzing, using a first electronic computing device, a plurality of words, wherein the second electronic computing device includes a processor and a memory, and organizing, using the processor of the first electronic device, each word in the plurality of words into one or more categories based on the analysis of the plurality of words. The method further includes receiving a plurality of words from the user, analyzing, using a second electronic computing device, the plurality of words from the user, wherein the second electronic computing device includes a processor and a memory, determining, using the processor of the second electronic computing device, which category or categories to which one or more words in the plurality of words from the user belongs, determining, based on the analysis of the plurality of words from the user and the determination of which category or categories to which the one or more words in the plurality of words from the user belongs, one or more psychological states of the user, and outputting, to a graphical user interface, the one or more psychological states.

It is an object of the present invention to provide the method for analyzing one or more psychological states of a user, wherein the plurality of words are derived from written or spoken text.

It is an object of the present invention to provide the method for analyzing one or more psychological states of a user, wherein the method further includes rating the one or more words in the plurality of words from the user within the determined category or categories.

It is an object of the present invention to provide the method for analyzing one or more psychological states of a user, wherein the one or more psychological states are selected from the group consisting of Intelligence Quotient (“I.Q.”); mood; thought content; thought process; and personality traits.

It is an object of the present invention to provide the method for analyzing one or more psychological states of a user, wherein the method further includes preparing, using the one or more determined psychological states, an assessment of any possible psychological conditions the user may have.

It is an object of the present invention to provide the method for analyzing one or more psychological states of a user, wherein the psychological conditions are selected from the group consisting of major depressive disorder; neuro-degenerative disorders, Alzheimer's disease; obsessive compulsive disorder; bipolarity separation anxiety disorder; attention deficit hyperactivity disorder; borderline personality disorder; and psychosis.

It is an object of the present invention to provide the method for analyzing one or more psychological states of a user, wherein the method further includes analyzing at least one string of two or more of the plurality of words from the user.

It is an object of the present invention to provide the method for analyzing one or more psychological states of a user, wherein the method further includes determining which category or categories to which the at least one string belongs.

It is an object of the present invention to provide the method for analyzing one or more psychological states of a user, wherein the method further includes determining, based on the analysis of the at least one string and the determination of which category or categories to which the at least one string belongs, one or more psychological states of the user.

It is an object of the present invention to provide the method for analyzing one or more psychological states of a user, wherein the second electronic computing device is selected from the group consisting of: a desktop computer; a laptop computer; a smart phone; and a tablet computer.

According to another aspect of the present invention, a system for analyzing one or more psychological states of a user is provided. The system includes a first electronic computing device, including a processor configure to analyze a plurality of words and organize each word in the plurality of words into one or more categories based on the analysis of the plurality of words, and a memory configured to store the analyzed plurality of words. The system further includes a second electronic computing device, including a memory, a graphical user interface configured to receive a plurality of words from the user, and a processor configured to analyze the plurality of words from the user, determine which category or categories to which one or more words in the plurality of words from the user belongs, determine, based on the analysis of the plurality of words from the user and the determination of which category or categories to which the one or more words in the plurality of words from the user belongs, one or more psychological states of the user, and output, to the graphical user interface, the one or more psychological states.

It is an object of the present invention to provide the system for analyzing one or more psychological states of a user, wherein the plurality of words are derived from written or spoken text.

It is an object of the present invention to provide the system for analyzing one or more psychological states of a user, wherein the processor of the first electronic computing device is further configured to rate the one or more words in the plurality of words from the user within the determined category or categories.

It is an object of the present invention to provide the system for analyzing one or more psychological states of a user, wherein the one or more psychological states are selected from the group consisting of Intelligence Quotient (“I.Q.”); mood, thought content; thought process; and personality traits.

It is an object of the present invention to provide the system for analyzing one or more psychological states of a user, wherein the processor of the second electronic computing device is further configured to prepare, using the one or more determined psychological states, an assessment of any possible psychological conditions the user may have.

It is an object of the present invention to provide the system for analyzing one or more psychological states of a user, wherein the psychological conditions are selected from the group consisting of major depressive disorder; neuro-degenerative disorders; Alzheimer's disease; obsessive compulsive disorder; bipolarity, separation anxiety disorder; attention deficit hyperactivity disorder; borderline personality disorder; and psychosis.

It is an object of the present invention to provide the system for analyzing one or more psychological states of a user, wherein the processor of the first electronic computing device is further configured to analyze at least one string of two or more of the plurality of words from the user.

It is an object of the present invention to provide the system for analyzing one or more psychological states of a user, wherein the processor of the first electronic computing device is further configured to determine which category or categories to which the at least one string belongs.

It is an object of the present invention to provide the system for analyzing one or more psychological states of a user, wherein the processor of the first electronic computing device is further configured to determine, based on the analysis of the at least one string and the determination of which category or categories to which the at least one string belongs, one or more psychological states of the user.

It is an object of the present invention to provide the system for analyzing one or more psychological states of a user, wherein the first electronic computing device is selected from the group consisting of a desktop computer; a laptop computer; a smart phone; and a tablet computer.

### BRIEF DESCRIPTION OF THE DRAWINGS

FIGS. 1-2 show a flowchart of a method for analyzing one or more psychological states of a user, according to an embodiment of the present invention.

FIG. 3 shows a system for analyzing one or more psychological states of the user, according to an embodiment of the present invention.

FIG. 4 shows a diagram of the general concepts of the Digital Psychometry (“D.P.M.”) method, according to an embodiment of the present invention.

FIG. 5 shows a flow diagram of Schema D4 (Existential Level Classification), according to an embodiment of the present invention.

FIG. 6 shows a flow diagram of Schema D7 (Kinetic Role), according to an embodiment of the present invention.

FIG. 7 shows a diagram of a schematic representation of text rhythm (Schema D11), according to an embodiment of the present invention.

FIG. 8 shows a diagram of Schema D17-1 (Self-Closeness Chart), according to an embodiment of the present invention.

FIG. 9 shows a diagram of a schematic view of a bordering (territory) chart (Schema 17-2), according to an embodiment of the present invention.

FIG. 10 shows a flow diagram of Schema E-1 (D.P.M. Software Model), according to an embodiment of the present invention.

FIG. 11 shows a flow diagram of Schema E-2) (D.P.N. Software Model), according to an embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

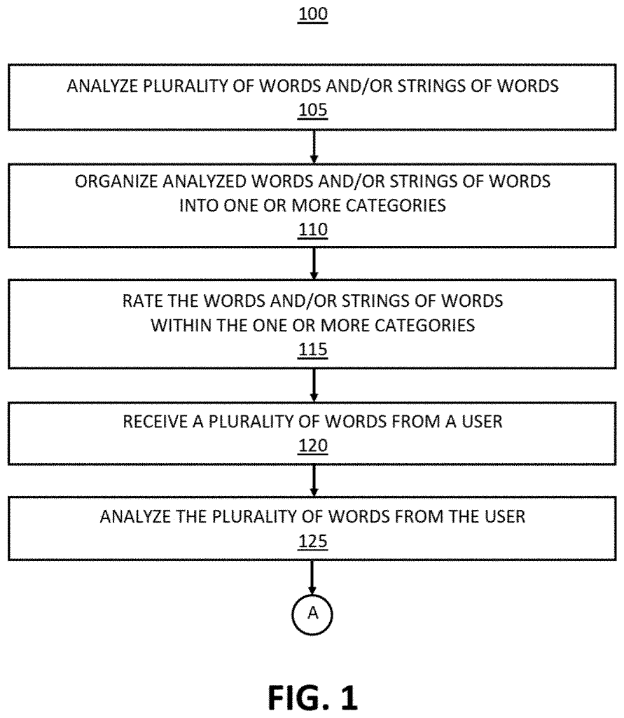

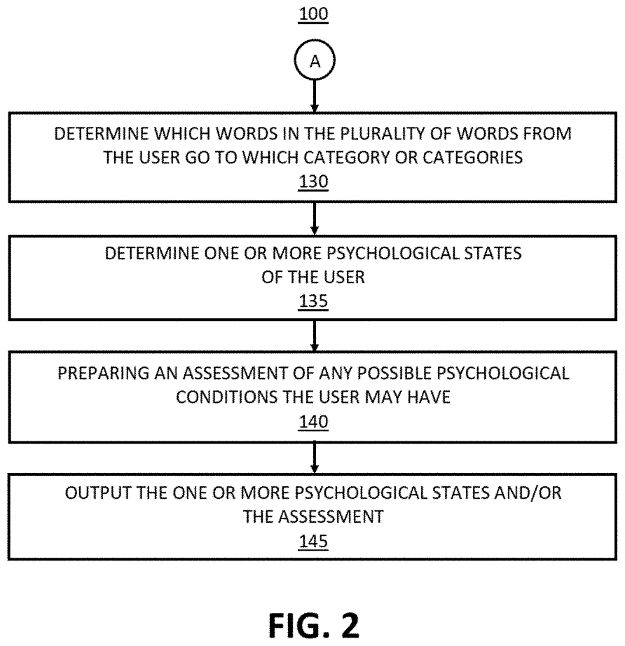

Referring now to FIGS. 1-2, a method 100 for analyzing one or more psychological states of a user is illustratively depicted, in accordance with an embodiment of the present invention.

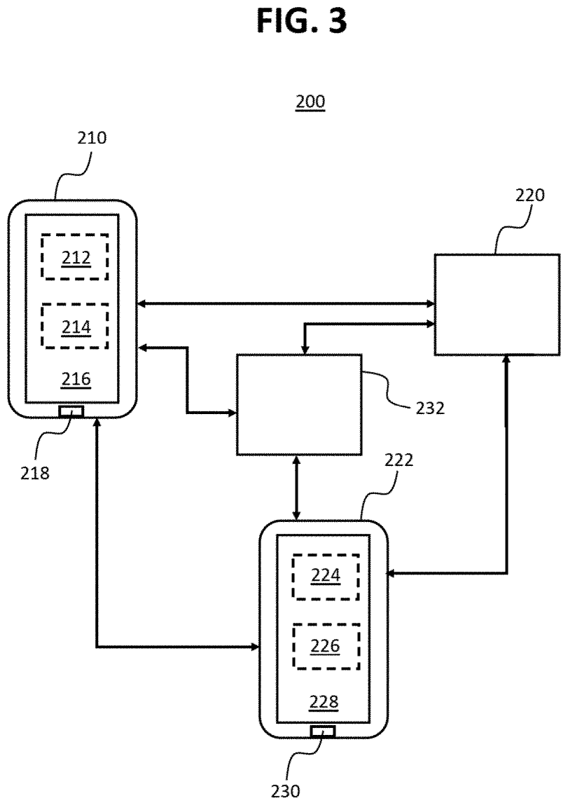

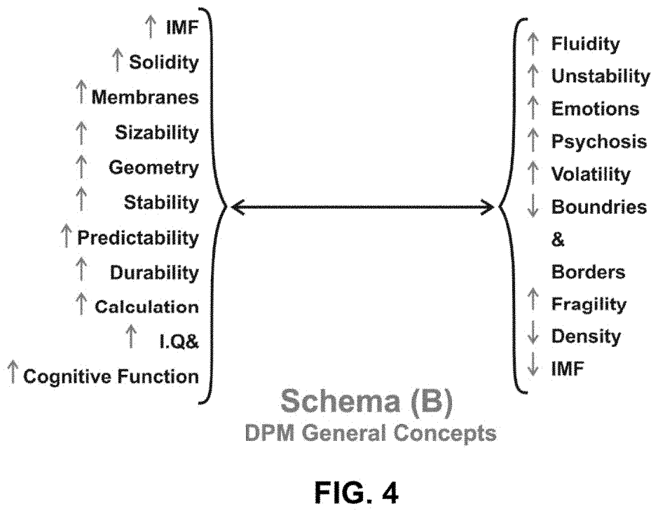

Digital Psychometry (“D.P.M.”) is a digital interpretation method of spoken and written texts. According to an embodiment, at step 105, using the D.P.M. method, almost all words are analyzed, regarding many aspects, and, at step 110, are placed into certain categories and, at step 115, rated. According to an embodiment, the analyzation and rating steps are performed prior to the beginning of the D.P.M. method. According to an embodiment, the analyzation and rating steps are performed as part of the D.P.M. method. According to an embodiment, this analyzation, categorization, and/or rating is performed using an electronic computing device 210 (as shown in system 200 of FIG. 3). This device 210 may include one or more processors 212, one or more storage devices/memories 214, a graphical user interface 216, and/or an audio recording device 218 (e.g., a microphone). According to an embodiment, the analyzed data is stored in the memory 214 of the electronic computing device 210. According to an embodiment, the analyzed data is stored on a remote storage device 220.

According to an embodiment, at step 120, a secondary computing device 222 (shown in FIG. 3) receives one or more words from a user. According to an embodiment, the secondary computing device 222 may include one or more processors 224, one or more storage devices/memories 226, a graphical user interface 228, and/or an audio recording device 230 (e.g., a microphone). According to an embodiment, the secondary computing device 222 receives the one or more words from the graphical user interface 228 and/or the audio recording device 230. According to an embodiment, devices 210 and 222 are separate devices. According to an embodiment, devices 210 and 222 are the same device.

According to an embodiment, the words may be spoken or written text. At step 125, the words from the user are analyzed by device 222 and compared to the analyzed words stored in device 210 or storage medium 220. According to an embodiment, device 210, device 222, and/or storage medium 210 may be coupled to one or more of each other directly (via a wired and/or wireless connection) and/or through one or more servers 232.

According to an embodiment, at step 130, it is determined which category or categories each of the user's analyzed words go in and these words are then put into one or more categories, and this ultimately leads to a swiftly and accurately cognitive, clinical, and analytical assessment of the text, as well as of the text's creator's mind. Therefore, the D.P.M. method, at step 135, can digitally provide invaluable information about the an individual's I.Q., mood, thought content and process, personality traits, and/or other psychological traits, and even give psycho-analytical point of views via inspecting the individual's own text messages.

According to an embodiment, at step 140, the system may further use the information to prepare an assessment of any possible psychological conditions the user may have. Also, according to these judgments, the D.P.M. method can go even further and provide the appropriate guidelines and prognoses to the individual in a more interactive way. According to an embodiment, one or more of the computing devices 210, 222 may be an electronic computing device and may be a desktop computer, a laptop computer, a smart phone, a tablet computer, and/or any other suitable stationary and/or mobile computing device.

According to an embodiment, at step 145, one or more of the determinations of the system are output onto the graphical user interface of one or more of the devices 210, 222.

According to an embodiment, the D.P.M. method incorporates the rating of the “words”, although on the next levels it can analyze and interpret phrases and sentences as well. According to an embodiment, the main work, as the text substrate, is on “words”.

According to an embodiment, the D.P.M. method can act as the missing part of artificial intelligence (AI), which is “Understanding & Interpretation” of human speech, as a prerequisite of devising understanding and interpreting software and robots. The D.P.M. method has actually stood on the junction of three complementary point of views which are mathematics, psychology and literature, to serve a better cognitive and psychological recognition of a person via his or her spoken or written text. This multidimensional perspective toward the words, however, is incorporated and integrated as a whole concept, according to human's mind by the inventor (who is an experienced psychiatrist). Therefore, the D.P.M. method is an integrating and formulating method of assessment, rather than a departing and dissecting one.

According to the D.P.M. method, it is postulated that “objects” have their own fundamentally genuine value, because the objects can be measured up (the math basis), pointed out as landmarks (the geometry & perspective basis), memorized (because they are more objective and more palpable; e.g., naming objects and figures is the first ability that is hampered by Alzheimer's disease), and they can also be more accurately predicted. Moreover, two major concepts of place and time are primarily shaped around objects, rather than other language elements. As a result, objects act as text cornerstones and almost all other text elements and grammatical components can evolve around them (including adjectives, which majorly shape around names). Therefore objects' names, which are frequently called Substance based. Objective and Sizable words (“SOS”—almost equivalent to concrete nouns), have been validated more than all other words, which are collectively referred to as “Non SOS”.

According to the D.P.M. method, it is also postulated that IQ, which strongly correlates with calculation and prediction, can shape around concrete nouns (SOSs), particularly solid ones. In contrast, “Liquescence and fluidity” is mostly concomitant with more emotionality, instability and psychotic states and less math and geometry abilities (creativity, however, often demands strong perspective making ability which is more categorized as a part of advanced geometry).

Hence, according to the D.P.M. method, it is preconceived that the solidity of a word is strongly correlated with its possession of distinct borders and membranes; whether it is a real object's name (concrete word) (naturally solid objects have detectable external borders with the outer space in which they are confined), or it is an abstract word for it a localization or an imagined external periphery could be conceived.

Therefore, in the D.P.M. method, the authenticity mainly goes back to “objects” (and, subsequently, concrete nouns), particularly solid ones, because they are much more measurable, predictable, and stable. Therefore, quantifiability, predictability, and stability are parallel concepts that have been tightly entangled with (the presence of) objects. As a result, the more that fluidity and liquescence lessens, and the more that solidity, integrity, and density (consistency) increases, the more the borders and membranes become much clearer and significantly sharper, the more mathematics and geometry become consequently much more emerged, and also the more it can be perceived that predictability, cognitive stability, and intelligence become proportionately more emphasized. The schematic diagram shown in FIG. 1 (Schema (B)) shows a part of these fundamental assumptions; that localization and the presence of membranes and borders are important concepts. These concepts will be discussed in further detail below.

According to an embodiment, the more the fluidity and liquescence lessens, and the more the solidity, integrity, and density increases, the more the borders and membranes become much clearer and significantly sharper, the more mathematics and geometry become consequently much more emerged, and also it could be perceived that predictability, cognitive stability and the intelligence would become proportionately more emphasized. The schema B (FIG. 1) helps to better understand these fundamental concepts.

According to an embodiment, the D.P.M. method includes two major components; the conceptual component and the software component.

The Conceptual Component:

According to an embodiment, the conceptual component includes all theorized discussions as well as all categories and maps that the D.P.M. method contains. Furthermore, according to the conceptual component, all of the words have fallen into distinct cognitive and psychological classifications (D1 through D28), which are described as follows:

D1) SOS & Word Structure

Word structure can be divided into two major portions; morphology and general appearance words, and structural words:

A) Morphology & General Appearance Words:

According to an embodiment, the morphology and general appearance of words refer to some external and more impressionistic characteristics of a word or object, including: shape; color; density; fragility; reflective ability; etc. These words are dominantly defined in connection with objects, but could be pertinent to the other words too. For further reference, refer to Appendix 1, the main table. The main table contains all SOS classification, including the morphology.

According to an embodiment, objects and, accordingly, all words can be rated based upon their general shapes (cavities vs. prominences), color, size, texture, density. Objects are also quantifiable, so they can also be classified according to their ability to be sized (angular, straight and solid ones versus rounded, curved and more liquid forms) and weighed. According to an embodiment, the object's potential geometric shape (triangle, quadrant, etc.), and also their shape resemblances to each other, can also be a base for sorting them out into certain categories.

B) Structural Words:

If a cellular-like structure for each object is conceived, then a better understanding can be had of how a word reacts with the outer space and whereabouts the main focal point and stress of the word is. According to this presumption, some main and some accessory components can be conceived for a word:

Object's Main Components (and the Related Words):

Word integrity is strongly related to the presence of these conceivable structures, particularly the core. Centrifugal words (e.g., proliferate; leak; dissemination), sacrifice this integrity for the sake of their outwardly communicative style. Fluidity, liquescence, and decreased Intermolecular forces (that are coming later) are pertinent concepts to the disintegration and the lack of the conceivable object's main structures.

1) The Core:

Many objects have a center and a core part and some words refer to a core and a central part, as well, and actually they have a central role (e.g., nuclear, pivotal, concentric); or at least are centripetal (e.g., alliance, solidarity, union, concentration).

2) Localization & Atmosphere:

Localization and atmosphere majorly refers to the context and the theme in which each object or word contains or refers. It becomes much more important when a word does not have any conceivable membrane or core. According to an embodiment, emotionality can be a part of it (e.g., bereavement, appraisal, sadness, dysphoria, hilarious, ecstatic).

3) Membranes & Borders:

According to an embodiment, words may have objective borders or at least refer to objective borders. Solid objects have distinct external borders. However, other words can indirectly remind us of the presence of a border, e.g., geometric words have a presumable border (consultation, captivity, transaction); and some words are pertinent to a perceived border (e.g., immunity, contact, protection).

To rephrase the concept, there are many abstract words that are directly related to the “structure” and the “integrity” which have been named in the D.P.M. method as “structural abstract words”; a portion of them are more static (“more statistically structural words”). Some of the more statically structural words directly refer to the “core” or the “base” (e.g., pivotal; central; basic; fundamentally; infrastructure); some of the statically structural words refer to the whole integrity (e.g., solidarity, consistency; integrity; organized); and some of the statically structural words refer to the borders, gates, and membranes (e.g.; ridge; limits; boundaries; entrance).

There are “more dynamically structural and integrity words”, too, that could strengthen or weaken these presumed structures; including Centrifugal words (e.g., stampede; widespread; dispersed), Centripetal words, which strengthen the core (e.g., focusing; reunion; repatriation; consolidation); border or the whole integrity strengthening words (e.g.; deepening; unitary; circumscribed; delimitation); and membranes and the whole integrity weakening words (e.g., dissecting; dismembered; decoding; infiltrating; influxes; leaking).

The importance of this overlapping categorization and subdivision mainly goes back to the presence and the degree of (integration) anxiety, fear, and the reaction formation that lies underneath these type of words; something that frequently is seen in psychiatric patients. Hence, the categorization of structural and integrity related words helps better the diagnosis of deeper layers of (integrity) anxiety done by the D.P.M. method.

Moreover, unstable and primitive personalities tend to destroy the structural words, not just by their behaviors but, firstly, by their verbal style; whether they are object names or structured abstract words by direct use of centrifugal, refraining, or disconnecting words; or by “pairing and mining” the structural words with deformity-making words and creating combinations like “smashing the records”; “revolutionary road”; “skyrocketed prices”; etc.,

Word Accessory Components:

1. SOS

The more a word retains more potential objects (particularly the specified ones), the more it is specific and quantifiable. In the D.P.M. method, it is hypothetically assumed that pragmatic and extroverted individuals strongly tend to use these types of words.

2. Place

According to an embodiment, the “place” refers to having a place component or a place relation. It can be a vague reference, e.g., homesick, patriotism, or it could be more specified, e.g., Eskimo, Irish, troglodytes.

3. Time

Time is a key element in words and gives the words more accuracy and increased functionality. Some word have an apparent time element, e.g., gradually, today, valentine, etc. For other words, nonetheless, the time element is more subtle. e.g., archeological, reincarnation, lazy, hesitancy, etc.

4. Word's Math & Geometry

According to an embodiment, the word's math and geometry implies the presence of the amounts, proportions, length, width, height, localized area, volume, and/or other quantifiable indices within a word. Therefore, it is significantly related to localization as well as having membranes.

Centrality Versus Marginality:

Many words can be sorted out according to their main stressed out structural region, whether it is the center or more inclined to margins. On the broader scope, words can be regarded as having a centripetal propensity versus a centrifugal feature for some other words, too. Centrifugal words like “rebellious, circumstantial, throw up, frantic” versus centripetal ones, like; “integrity, merciful acceptance, pivotal, concentric”). Additionally, words can be regarded as having convergence versus divergence, two terms that are indicative of the distances among the objects. According to an embodiment, the main implication of the centrality and marginality concept would give more insight in the recognition of a person's mentality, whether it is more center and self-oriented, more static and more integrative, or, on the contrary, it is more outgoing, more kinetic, and more chaotic; or it is paradoxically a mixed type (as we can see in personality disorders).

Word's Orientation

According to an embodiment, a word's orientation is brought under the category of connectivity. It is important to know the difference between time- and place-oriented words and those who lack these stabilizing elements. Many words directly demonstrate a time element (e.g., date back; futuristic; historical); while there are other words that carry an insidious time element (e.g., preparation; sunrise; reconstruction) There is a similar comparison for place-bounded words as well. According to an embodiment, there are words that imply a place (e.g., seaside; Londoner; western), and those that carry a subtle place and geography element within themselves (e.g., patriotism; deforestation; departure). Time and place oriented words are considered to be more specific, more functional, and take higher marks in terms of stability in the D.P.M. system, too.

Word Specificity

Word specificity is a fundamentally important concept. Word Specificity is defined as having one of these features: 1) The presence of distinct SOS(s) within the word; 2) The presence of Place element(s); 3) The presence of addressing & directional words; 4) The presence of Time element(s); and 5) The presence of detectable Math & Geometry.

D2) Vitality Elements & Libido:

Libido can not only be seen in the object's shape and kinetics, but also can be recognized by the presence of vitality elements, including water, greenery, heat and the biological component (as seen in adult genitalia). All the objects can be classified according to the libido; regarding either the presence of each vital elements or their globally structural or functional resemblance to human's genitalia; or their insidiously inner libidinal value (money; jewelry; hunting stuff, gun, etc.).

By rating the objects as well as “all of the other words” (whether they contain SOS or not) according to their libido, the “summed-up libido” and “vitality degree” of a text, and those of its creator's mind, can be “digitally” rated and charted and revealed in the “Libido chart”; hence, it would convey a better diagnosis of some psychiatric disorders in which the libido has remarkably changed. Libido is ramped up in some mental disorders, such as bipolarity, as well as some primitive personalities, such as antisocial personality disorder. In contrast, libido is significantly decreased in some others, such as in patients involved with major depressive disorder or some neuro-degenerative disorders such Alzheimer's disease.

Rating the vitality degree of a person should not necessarily get related to having an illness and/or disorder; many lovers are keen to know how much desire and infatuation is truthfully invested in a romance, by their own lover and how much chemistry exists in their relationship, particularly a long-distance relationship.

D3) the Word's Primary Spatial (Physical and Social) Role Classification

When an object moves in the space in which it is confined, it moves in several distinct pathways. For example, the object may move freely and independently, according to the laws of physics; it may become more interactive with time, place, and, more importantly, with other space objects; it may get old, or eroded; and, finally, it may get disrupted, dissected, disconnected, and torn-off. Therefore, spatial movements and, accordingly, the spatial roles, whether they are more individual or more interactive, fall into several certain categories that include: Existence roles; Equilibrium roles; Stability & Integrity roles; Kinetic roles (more independent and free movements); Action level roles; Contact & Connectivity roles (more interactive and goal-oriented); and D/C roles(Dissecting, Dividing. Deformity making).

It is important to know that, when a person writes or speaks, their choice of words and, particularly, “their choice of verbs”, has been selected from the above-mentioned categories; because it can be subsequently linked to some quite specific personal states or traits. Words can have geometrically distinct and classifiable roles. These roles can be dominantly physical and individual roles, or can be more social roles. The major role that a word can take in the outer space is surprisingly limited to the distinct categories mentioned previously. Classification of words in accordance with their spatial position not only helps to get the people's point of view toward a subject, but also can disclose many psychological facts about them. For example, a person who is frequently applying some “dominant role words” may have some authority-related conflicts; or an individual who is consecutively administering “disintegration words”, may have some degrees of integrity anxiety.

Since words can play more subtle physical, social, and/or psychic roles throughout a text; the scrutiny over these minor roles can also give invaluably delicate information about people's subconscious thoughts and feelings. For example, in the following sentence, there is a hidden sense of feared contact by the speaker; “It has been 2 years since he has arrived here as an apprentice and has faced many challenges in the workplace”. Because the words “arrived”, “face”, “apprentice”, and “challenges” all have a “contact” role particle, while the word “challenges” has some negatively struggling and interactive role, it can be deduced that the writer of the sentence is an obsessive-compulsive person. Although the psychic role can be complementarily brought with physical and social roles (as a part of a major geometric scheme), it has been brought separately in order to avoid any possible confusion.

It is noteworthy that a word can have more than a sole physical role or a sole social role. Therefore, the word can be incorporated into more than one category at the same time.

Besides in the D.P.M. model, the degree and the severity of the physical and social roles has been ranked and described as low, moderate, and severe. Therefore, in this system, not only is the type and the role of applied words qualified, but also the severity and their extent of happening can be digitally determined, too.

It is noted that some more sophisticated social roles have not been included here. D4 to D10 discusses these major spatial roles in more detail.

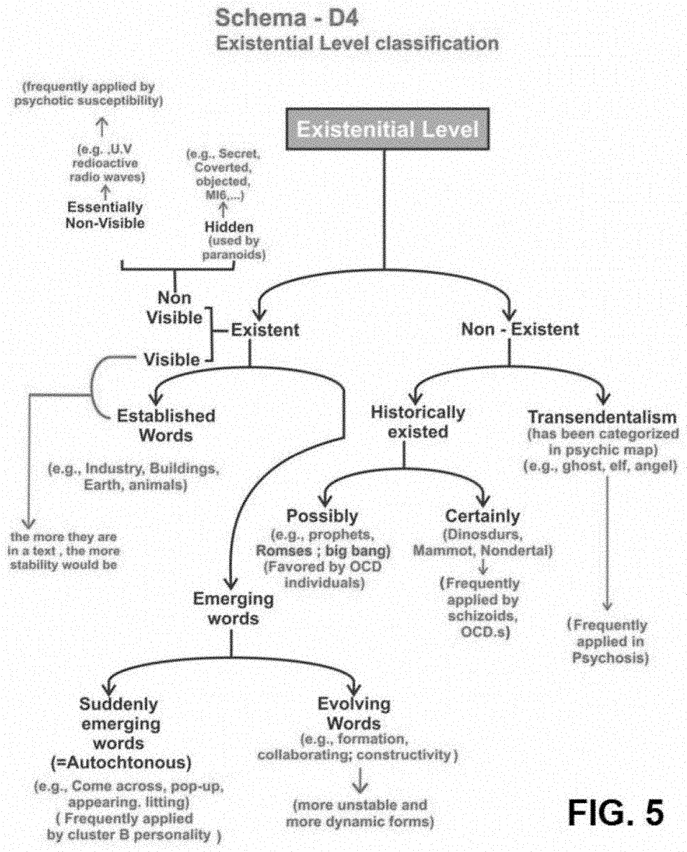

D4) Existential Level (Main Table/Schema-D4) (as Shown in FIG. 5)

This more primitive and less geometric feature has some linkages with structural aspects of an object or a word on the one hand, and some linkages with some more dynamic and functional aspects of it on the other hand. For example, the lack of distinct membranes for a substance or conceivable borders for a word is more inclined toward the structural aspects of the existence (e.g., ghosts; daemon, radioactive). In this regard, loss of a word's borders, which can be observed in heightened emotions, as well as classical psychoses like schizophrenia, can be adequately referred to, while considering the sudden and autochthonous emergence of an act or an object goes back to more dynamic aspects of the existence (e.g., formation; flare up; appearing; occurred). According to an embodiment, history and chronology mostly refers to the existential roles (because they frequently refer to a clear-cut happening of events). Therefore their content may get less functional grades in the D.P.M.

According to an embodiment, there is a schema here for the better understanding of this concept (Schema-D4). Besides, the existence possibilities have been separately referred to on the main table. Regarding the existence concept as a part of spatial deployment of words and trying to detecting it throughout a person's speech, this method remarkably helps to better trace primitive thinking, chauvinism, cultural psychoses, and even schizophrenia.

D5) Equilibrium Level

Some words are specifically dedicated to express a sense of balance and equilibrium in the space; and are more frequently applied by those persons who have some issues in this regard. Borderline personalities frequently use words that are directly related to the sense of balance or ataxia (e.g., relying; mainstay; lurched; symmetrical; dizzy; suspending). Similarly, obsessive-compulsive individuals frequently administer those words that mainly refer to a vertical balance (e.g., vibrating, basically, bearing; upraised).

D6) Integrity/Stability Level (Main Table)

Integrity of a word goes beyond its existence and it also belongs to the words that are mainly related to the sense of solidity, cohesion, and attachment (e.g., strengthen; consolidate; unitary). This category, even in its mostly positive way of expression, can be a form of “reaction formation” (a defective defense mechanism seen in neurotic persons), concealing a fear of disintegration and collapse on the inside; therefore, applying these types of words raises serious doubts about the expression of the real and genuine feelings and intents of a person.

Similarly, the words that are indicative of detachment, divergence, and division, can be categorized under the title, too (e.g., subversion; dissociative; decay; break-up; collapse).

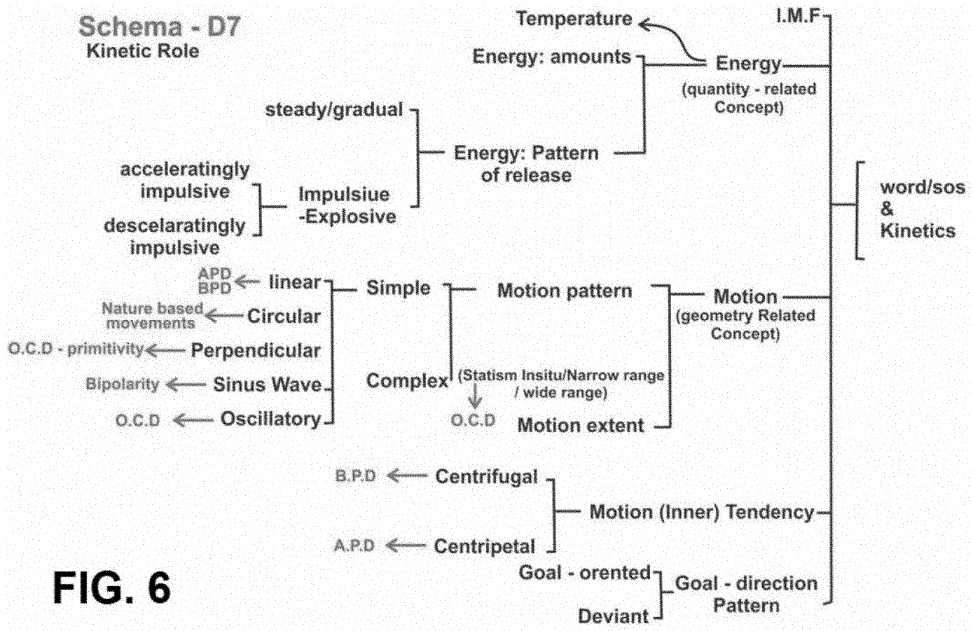

D7) SOS and Word Kinetics (as Shown in FIG. 6)

This term entitles the whole range of kinetic-based concepts, including Intra-molecular forces (IMF), Potential Energy, and also some more geometrically based motions including: Motion Extent, Motion patterns, velocity, etc., that have been referred in the schema D-7

IMF (Intra-Molecular Force):

Implies the binding capacity of molecules within the object. It can be generalized to all of the words. IMF is strongly pertinent to the “fluidity concept”, and also to the volatility of an object. Slumped IMF can be psychologically related to some heightened emotional states, as well as to an increased risk of psychosis.

Potential Energy:

This concept can be also categorized as a part of word and object kinetics. It can be inspected regarding the amounts, as well as the major pattern, of energy release. The former one mainly goes back to libido; while the latter is mostly related to stability and personality qualities. For example, a sudden and abrupt pattern of energy release is majorly seen in borderline personality disorder literature speech, revealed by their subconscious choice of impulsive and explosive words abundantly applied in their own literature.

Centripetal Vs. Centrifugal Words:

They refer to the tendency of words to be more core-oriented or, in contrast, to be externally oriented words, whether these words are concrete (SOSs), or, reversely, abstract words, verbs, infinitives, or gerunds. Stability or dynamism of a text to some extent goes back to the centrifugal (e.g., redemption; outspoken; disseminating) or centripetal (e.g., pivotal; repented; fixation; unanimously; concentric) tendencies of its constituent words.

Motion Extent:

We can classify all the objects and words according to their (potential) range of motions, too. These include Statism. In situ movements, Narrow-range motions, and Wide-range motions, respectively.

The range of motions has also some connections with particular mentalities. For example, static and low-range of motion words (e.g., scrutiny; hesitation; precaution; introverted); are frequently applied by Obsessive Compulsive Disorder (OCD)/SAD (Separation Anxiety Disorder) persons who are mostly territory-bound while the words with a wide and free range of motions (e.g., flight; widespread; speed up), are dominantly administered by bipolar individuals.

Motion Patterns:

Although there are numerous patterns of movements, they are categorized, then it can be witnessed that people choose their type of movements in the outer space according to a repetitively stereotypic pattern, and according to their personality. For instance, simply and shortened linear patterns of movements are majorly favored by primitive personalities, like the personalities of psychopaths and borderlines; “bypassing words” are the example of such a spatial movement, while “oscillatory movements” (and, of course, the related words) are frequently exploited by OCD persons as well as ADHD individuals and tic disorder individuals. Bipolar persons strongly tend to use sinus wave patterns of movements. Verticality patterns of movements, nonetheless, are majorly observed in primitively as well as OCD.

According to the direction tendencies of the movements, words can also be divided into centrifugal and centripetal Centrifugal words are one of the favorite cluster of words applied by bipolar persons. Obsessive persons and dysthymic individuals, however, are more center-oriented in nature (and accordingly more stable, too).

Therefore, categorizing the words with this regard helps to procure a better diagnosis of the text writer.

D8) Receptiveness—Action Level Spectrum

It is important to inspect all of the verbs and their own derivatives (infinitives, gerunds) in a text, regarding their level of receptiveness and passivity on the one side, and the action level on the other side.

By applying this index, it can be estimated whether a text owner is insubmissive, solitary and schizoid state, or in contrast is dominantly interacting, motivated, and socializing toward the others. A classification of the first side of this spectrum, which is receptiveness, has been brought here in ascending order, skipping the high action-level classification, which will consecutively come. This receptiveness classification, in ascending order, includes: Avoidance words; Sensory-Perceptive words (except for visual sense): Acceptance words; Inwardly mood words; (Outwardly)Visionary view; Outgoing Thoughts; Outgoing Mood words; Intent level words; Capability & Esteem words; Vigilance words; and Permission words.

D9) Action Level

It indicates the degree of the action that verbs, and their own derivatives, carry within themselves. It substantially differs from word kinetics, since the latter refers to more individual and free aspects of movements, while the former (as its name stands for itself) mostly refers to the interactive and social aspects of the objects movements.

We can have the following hierarchy according to the level of action (in ascending order): Action plan level (Intent level); Capability & esteem words; Transitive verbs; Prescriptive words; Inductive words; Manipulative & deformity making words; Explosive words; and After-burn words.

According to an embodiment, there always should be a proper balance between a text action level on one hand, and the presence of receptive and thoughtful words on the other hand. Functionality depends on the presence of this equilibrium, seen throughout a person's talks. High action level can be witnessed in some personality matters, like borderline and antisocial personality traits and disorders. There is no doubt that a vast difference exists between action level and connectivity level; since the former, owns a less constructive style, with a lower control factor (detected by the so-called chart), and the latter is a major indicative of highly function. Hence, by recognizing the action level the susceptibility of acting out, aggression, and violence can be preventatively and digitally predicted by D.P.M.

D10) Contact & Connectivity

The ultimate goals of speech are continuity, communication, and connectivity. Therefore, all the words (including the verbs) can be categorized and valued in terms of this basic concept, as it is done in the D.P.M. model.

“Continuity” refers to the words and also phrases that carry a continuation or elongation meaning on the inside. In other words, they semantically depict a line, or insist to elongate it (e.g., in accordance with, living, pursue, goes on, evolution, ensuing, along with). On the other side, we can refer to “D/C words”, a broad cluster of words including misalignment words (e.g., disarray, derailed, pervert), detachment & disintegration words (e.g., break-up, shattered, falling apart), Impulsive words (e.g., suddenly, shut up, kicked in, rushed into, cut-off), explosive words (e.g., blew up, burst into, blurted out), suddenly emerging or disappearing (autochthonous) words (e.g., pop-up, hidden, clear-up, vanishing; emerged from), etc., which reveal interruption, disruption, and discontinuity. Clear-cut words (e.g., crossing, start up, get-out) are supposed to be a part of this side, too.

“Connectivity” is another similar concept that, because of its great functionality values, has been discussed and classified dividedly. It generally refers to the (fine and qualitative) binding capacity of words, to serve the text cohesiveness, or to depict a larger geometrical shape by means of the words. To clarify, words resemble pieces of block toys, but with more variations in size and shape, as well as much more flexibility. They can be chaotically put together, they can make simple linear shapes (trains, wall, etc.,), and/or, in the most sophisticated forms, they can precisely and finely attach together to form an artistically created sculptures. Similarly, connectivity of the words can make simply linear geometry, or in it's mostly advanced form build a symmetrically geometrical shapes throughout the whole of text, which is herein referred to as “The Hexanoids” (discussed in D4).

Connectivity strongly correlates with math and, therefore, with mental functionality. To explain, in the D.P.M. method, it is conceived that the ultimate goal of any written or spoken text is “communication and connectivity”. Therefore, each word, as well as the whole text, should be scrutinized with respect to this ability. This is regarded as the undeniable aspect of functionality.

Connectivity and continuity are essential concepts that have been regarded as a basic index in D.P.M, for categorizing and also quantifying the words. In terms of this, two distinct indices have been defined by the names “Connectivity Value” Index and “Continuity Value” Index, both briefly referred as “C.V. index”, or “C.V.I.”. Therefore, each word can receive a C.V.I., which represents its binding affinity to other words, or represents the continuity of an act (we can also name the continuity words as “the range words”). However, the C.V. index mostly represents the amount and the quantity of connectivity affinity of a word. Another index has been defined that mostly stands for the “quality, sophistication, and fineness” of connectivity, which is named Fine (or full) Fitting Index (F.F.I.), is only applied for connective words (not like the C.V. index, for all the words). For example, the words “amalgamation” “interaction” and “combination”, have higher C.V. indices but lower F.F.I.; while the words “Wiring”, “pairing” and “match fixing”, vice versa. Since continuity and connectivity serve to build up lines and alignments, as “the precursors of geometry”; hence, it can be assumed that each word that complies for this alignment capability in a stronger or better way, has more C.V index, or F.F.I, respectively.

On the opposite side, the words that have the capability to interrupt these alignment making capabilities, including D.C. words, misalignment words, chaos making words, disintegration, and deformity words, contradict continuity and connectivity concepts. They essentially hamper the text cohesiveness, as well as it's “geometry”.

Concrete words (SOS) connectivity capability can be “structurally” related to having loose membranes, wet surfaces, or lower I.M.F (Intermolecular force), while abstract words (NON-SOS) have more intricately structural aspects. In its mostly primary form, connectivity of a NON-SOS form includes a range from two objects that colliding, or merging with each other (e.g., symbiotic, blending, mixture), to in its mostly advanced form which is a smartly directed communication (having an awareness factor, too) among two objects or persons who are interacting through an objective hub, and by means of a transitional object. (e.g., phoning, shipment—these words apparently receive higher F.F.I). All other forms of connectivity can be placed somewhere in between. In order to understand the concept better, the main table reveals all types with some examples.

It is noteworthy that the connectivity types of words, which paradoxically contain an insidiously disconnecting or disruptive inner element (particularly colliding and oscillatory types that are extensively used by borderline personalities with O.C.D. comorbidity), have, therefore less connectivity values (C.V.) than the other connectivity types like permanent contact ones. For example, the words “twinkling, collision, accident, flickering, lit up, boxing, and fluctuations” have less connectivity values than the words “sticking, symbiotic, annexation, union, and combinatorial” because they bring to mind a sense of temporary attachment. Accordingly, not only the verbs, gerunds, and infinitives can be classified with respect to their connectivity values, but also “objects” can be categorized in this regard. For example, the word “knife” has less connectivity value than the word “belt”.

Connectivity has some more “dynamic” aspects, too. It can include: Classical connectivity (which includes 1. Glance/Nice touch (e.g., tangible; snapshots; touching on); 2. Intermittent-Oscillatory contact(e.g., flickering; knocking on tapping); 3. Durable contact (e.g., Marriage; annexation; breast-feeding)); Transferring Connectivity (e.g., capitalizing; transactions; fueling; delivering); and Interactive Connectivity, which includes Positively interactive connectivity (e.g., negotiations; gathering; collaborative) and Negatively interactive connectivity (e.g., discussions; struggle; disputes).

In summary, according to connectivity and communication properties, a spoken or written text can be assessed word by word and/or on aggregation, to define how much a text is cohesive and integrated and to what extent its words are finely and sophisticatedly matched. Any disruption in terms of this could convey the text creator's disorganized speech, as well as an interference of a mental disorder.

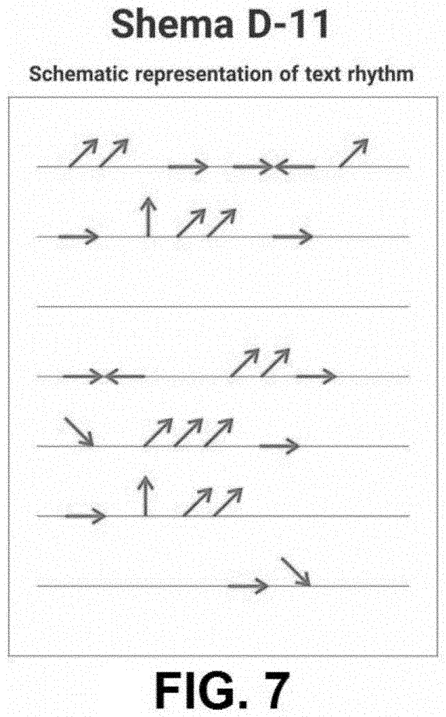

D11) Word Angle (W.A.) & Word Directivity (as Shown in FIG. 7)

This concept is originally regarded as a part of word & text geometry. However, because of its fundamental importance, it is herein separately described. According to an embodiment, words can have “angle” and “direction”. These are two overlapping yet different concepts that have been unanimously discussed herein.

Along with the lines and extensions, angles and directions are the major precursors of text geometry. This concept can convey to other major concepts, like “text rhythm”, “text harmony”, and, finally, “Hexanoids”. Directions can be essentially pertinent to the “object”, “time” or “place”.

Majorly related directions toward an object include, but are not limited to: anterior, posterior, medial, lateral, superior, inferior, and their more sophisticatedly combinations, such as antero-lateral, postero-medial, etc. Directions also can be classified toward the surface, which includes outward/outer/external, inside/inner/internal, on the surface/superficial.

Objects cannot only take a stand, or move according to these directions, but can also have an in-place axial rotation and spinning around their axes, as well.

Some other sectional directions toward the objects can be secondarily pertinent to objects, like, coronal, sagittal, and longitudinal sections. Directions can also be linked to “place”. North, south, west, east, and their more cognitively advanced combinations, like northwest, southeast, etc., are examples. “Left and right” are also more place-bound words, rather than to be object-bound. Longitude & latitude are much more place-related concepts, as well.

As mentioned above, directions can be also relevant to time. Past, present, future, and their more importantly tense (past-continuous, past-participle, etc.) subgroups are indicative of the tense-related directions.

Directional words can be categorized in three groups:
1) Purely directional prepositions & words.
   a. Their existential philosophy is revealing the direction but no other reason (e.g., Northwest, outwardly, inner).
2) Direction-containing words.
   a. They reveal potential or real happening of an event in a directional and angular way; e.g., descent (↓), balloons (↑), skyscrapers (↑), slumped (↓), capsize (↓), resurfaced (↓), etc. Accordingly, gradually decremental or incremental words can be assumed that carry within skewed angles (they are very important, since they represent relative words); e.g., increase ( [symbol] ), evolutionary ( [symbol] ), descent ( [symbol] ), escalating ( [symbol] ).
   b. Direction-containing words sometimes have more subtle angles. For instance, according to an embodiment, Entrance-exiting words (e.g., welcome, newcomer, welfare, etc.) can be assumed to have 90 degrees angles toward the object, while simply linear or parallel movements can be assumed to have zero degree angles (e.g., accord; continued; moving forward; going on, etc.). Some others are more combinatory. For example, “surfing on” represents a vertical and dominant positioning, while having a simultaneous horizontal direction, too.
   c. There are more subtle angular words, too. “Oppositional words” can be regarded as having 180-degree angles within (e.g., abhorrent; counteroffensive; appalling; hatred; battle; conflict; contravene; counterbalance; counterproductive; confrontational backfire).
3) Conventionally directional words.
   a. This category is based upon the assumption that a conventional angle for some words can be presumed, according to the amounts of their ingredients.
   b. Full amounts of any type (severity/speed/weight, etc.), which are reminiscent of some fully erected structures, presumably have 90 degrees.
   c. Partial amounts are considered to have 0-90 degrees, while opposing and negative amounts have more than 90 degrees. (“accordance and parallel words” are considered to have 0 degrees). Accordingly, a conventional angle can be given to severity (mild, moderate, severe), too.

### What are the Practical Implications of Such a Classification?

By recognizing the word angles and directions, whether it is real or conventional, the more fundamental concepts, including “text rhythm” (Schema-D11), “text harmony”, and “Hexanoids” can be reached, and they will be discussed here:

With regard to the directivity aspect, an incomparable chart (“Text Rhythm Chart”), which can reveal how a person tries to create a rhythm through his or her harmonious speech or text, or, in it's mostly sophisticated form, portray a umifying geometrical shape by his or her own words in the outer space (Hexnoids). This chart (like the DaVinci chart and the Negativity-Positively chart) can swiftly represent invaluable information about people's trends, their inner inconsistencies and conflicts, and their personalities, since the patterns of movements in the outer world can be idiosyncratic, too.

This chart can not only reveal the summed up “text angle” and the “text harmony”, but can also demonstrate any potentially geometrical shapes that can develop within a text by collective and collaborative presence of directional and angular words (those polygonal shapes that are named by the inventor as “Hexanoids”).

Hexanoids (if they exist in a spoken or written text) are considered to be the hallmarks of highly functional and highly cohesive texts. Hexanoids are shapes that have been developed throughout a text by the simultaneous applying of specific nouns and directional words in order to illustrate a holistic and incorporative geometry. However, they only can be seen in some highly functional texts, such as scientific texts, as well as poetries.

Text rhythm, text harmony, and Hexanoids can be schematically illustrated by the computer in the D.P.M. method via a “Text Rhythm chart”. These structures, particularly Hexanoids, are indicative of a person's cognitive ability of adopting geometry in order to visualize a perspective through his or her own speech.

More importantly, the pattern of choosing angles and directions by a person can have some connections with some psychological states, as well as traits. To explain, some instances have been referred: paranoia has some linkages with posterior (behind, back) direction; and verticality (upward, downward) is frequently used by obsessive-compulsive persons. There is a major Retrospective and past tense trend among dysthymia as well as OCD, and S.A.D. individuals (nostalgia, homesickness). A sense of apprehension toward (a shortened) future is usually seen in PTSD cases. Ataxia, vertigo, and all relevant words which are pertinent to rotating and leaning directions, are frequently seen in borderline personality literature. Outward positions and penetration is majorly witnessed in highly pragmatic persons. On the contrary, inwardly penetrative style is frequently applied by borderline personalities. Less cognitively developed brains and lower I.Q. individuals less often tend to apply two-dimensional or combined directions (antero-medial, northwest, sagittal, longitude & lassitude, etc.) in their own words.

D12) Word Math & Geometry

Words can have an inner math, direction, and geometry. Human speech contains mathematics (and, in more advanced form, a geometry), which can be smartly extracted and digitally measured, as is done in the D.P.M. method. Any spoken or written text can thus be quantified and qualified in terms of the inner mathematics and geometry. Subsequently, a reasonable estimate of the person's cognitive ability, including the intelligence, can be acquired. This is an unprecedented and unheard of perspective toward words and speech.

There are three main levels of math assessment in a text:

1) A word's individual math, angle and geometry: refers to a word's inner math, angle and geometry, which has been devised for almost all common words previously quantified.

Word's Math Grade (WMG):

It is a concept that each word has to have an associated grade of math, according to the level of math and the geometry it carries. Therefore, not only can the type of each word's math be determined by means of this grading, but also the collective and quantitative math grade of each text could be consecutively estimated.

2) Collective (amounts of) words math, angles and geometry: It is the quantitative and collective summation of all the text words' individual math, angles (text rhythm), and geometry. It is mainly related to the amounts, rather than to the whole picture.

3) The text's holistic geometry & Hexanoids: It implies a holistic, unifying, and cohesive geometry that exploits almost all the words of a text to create a comprehensively portrayable geometry, which is herein referred to as “the Hexanoids”. Not all the texts contain this cohesive geometry; (the so-called hexanoids).

Four subjects can be the matter of mathematical and geometrical measurements within a text. These four subjects are:

1. The SOSs and places names: SOSs can be measured up with respect to their weight, size, and/or altitude, or can be geometrically evaluated regarding their symmetry or asymmetry and also their own globally geometric shape (triangle, quadrant, etc.). Places can also be measured up, regarding their area, their altitude, etc.

2. Movements and acts: This subject also can be calculated by quantitative math (the velocity and acceleration) or by geometry, whether it is inside the word which is called word geometry or it is pertinent to verbs and their own spatial patterns of happening (the movement range, pattern, etc.,), or the negative spaces among the objects. The word angle can be a part of word geometry, too. Action nouns and gerunds can also contain word math and geometry which can be revealed and measured up by the D.P.M. method.

3. The word state-geometry: Many words have a “within” component of geometry which can be extracted, depicted, and qualified. For example, words such as “captivity,” “straddle,” “narrow down,” “polygonal.” “stratified,” “withdrawn,” and “queuing” are geometric words. This means that these words have their own “within” geometry, which can get drawn and depicted.

Recognition of these internal geometries is a part of word decoding, done by the D.P.M. method, which helps to formulate a better understanding of the semantic associations of a word with its neighboring words in a paragraph, or within the whole text.

4. Connectivity geometry: “Text rhythm”, which is the summation of all the word angles and directions in a text, can be as a part of this holistic geometry. In the most advanced form, the geometry of each word serves to create a bigger picture which has a more generalized geometric shape in a paragraph or in the whole text. Therefore, holistically geometrical patterns can be seen in each paragraph or within the whole text, which are referred to as “the hexanoids”. Hexanoids can be only seen in quite organized and geometrically illustratable texts, including some scientific texts or in some poetries.

The Math Evolutionary Classification

There are many types of math and geometry which have been schematically brought in the schema-D12. The schema has comprehensively referred to these types, according to their sophistication level (in descending order). There are many types of math regarding their level of evolution and advancement, as it has come in the above mentioned scheme.

Each of these components has its own literature and vocabulary. For instance:
Absolute amount math: (e.g., 1978; 25 kg; 30 megabytes)
Proportional math: (e.g., a fraction of half; multiplying; two fold)
Incremental math: (e.g., doubled; skyrocketing; accelerated)
Decrement math: (e.g., portioned; slumped; downgrade; fragments)
Linear Geometry: (e.g., in accordance with; followed by; drawing; alongside)
Derailment Geometry: (e.g., deflection; disarrayed; pervert)
Parallel Geometry: (e.g., parallel to; concomitant; comorbidity)
Distribution Geometry: (e.g., prevalent; incidence; localized; dissemination)
Plane Geometry: (e.g., areas; spread out; superficially)
Triangle Geometry: (e.g., collusion; circumvent; treacherous; embezzlement)
Dual Geometry: (e.g., romance; break-up; tennis)
Negative Space Geometry: (distance; amongst; disparity; discrepancies)
Solid Geometry & Perspective: (e.g., accentuate; ante-lateral; sagittal; multidimensional, insert)

Moreover, the more the math levels become more advanced, it is perceived that the cognitive ability of its applier would be subsequently more sophisticated. In other words, the inner mathematics and geometry of the words are strongly correlated with the user's I.Q., particularly when the amounts and the type of math are contemplated approximately simultaneously. Some geometry-contained words can be difficult to understand for lower I.Q.s; i.e., a moderately mentally challenged individual would likely not understand the meaning of words such as “extradition”, “captivity”, or “recapitalization” well, even after being provided with sufficient explanations, but he or she can likely easily understand emotionally expressive words, such as “anger” or “fatigued”, which lack the sophisticated geometric elements.

Some examples have been briefly brought here to provide a better understanding of the types of math and geometry. These examples include:

1. Iran has been placed in the center of Middle East in the Asia continent. (simply centrally spot geometry)

2. The more we go toward north in Iran, the more humidity we would be encountered. (Parallel geometry; co-direction type)

3. The more we move southern-ward, the less the greeneries would be seen. (Parallel geometry contra-direction type)

4. Iran has 6840 kilometers borders in total. (Absolute amount math the most primitive form)

5. Iran has water borders as well as dry borders. (Simply splitting geometry)

6. Iran has equally balanced water and dry borders. (Equation math)

7. Iran's northern borders is larger than southern one. (Comparison math—simply static type)

8. Iran is the second largest Middle East country. (Level math)

9. Iran has an area about twice as Iraq. (Proportional Math—Incremental type)

10. Iran lies between Caspian lake and the Persian gulf(Simply Linear Geometry)

11. Iran has been peripherally surrounded by Iraq, Turkey, Armenia, Afghanistan and Pakistan. (Bordering Geometry)

12. Iran has borders with Turkey and Iraq on one side and Afghanistan and Pakistan on the other side. (Centralized Parallel Geometry)

13. Iran in northwest is limited to Azerbaijan, in central north to Caspian lake and in the northeast to Armenia; it is limited westerly to Turkey in the above, and Iraq at the below, while in the opposite side Afghanistan and Pakistan are placed respectively. Central southern parts of Iran is limited to Persian gulf while in southwest and southeast, Saudi Arabia and U.A.E, have been settled respectively. (Hexanoid Geometry—a fully polygonal shape)

What are the Practical Implications of Recognizing the Words, Text Math, and Geometry?

A) Assessment of the Person's Cognitive Abilities & Intelligence

By quantifying the amounts of the applied math and geometry within a text, and also by qualifying the type of the administered math and geometry, a person's mathematical mind can be effectively unraveled, diming to get invaluable information about his or her cognitive abilities as well as I.Q.

The more the math and geometry are sophisticated and advanced, the more the cognitive ability of a person would be. It is noteworthy that, in contrast with literature that exerts numerous ways of expression, the underlying math and geometry is quite constant and inalterable in nature, which can be recognized and cognitively judged.

The type of applied math and geometry in a text can also reveal the visualization capability, the mental stability, the academic function of their owner, as well as some accurate estimations about the I.Q. of the person, i.e., someone who uses a simply linear chronology in his or her speech, cognitively is superior to someone who is applying impulsive and explosive words, but is cognitively inferior to a person who is administering more intricately geometrical words and more perspectives during his or her speech.

This is an extraordinary capability of the D.P.M. method. Here has been brought another example to express how the D.P.M. method can recognize the insidious geometry throughout a spoken or written text: “ . . . with a monotonous tone, the tail of heavy wooden cross was continuously drawing a line on the soil. Someone screamed; put the prickle crown on his head! . . . the scratching sound of the cross was weaving with his painfully delusional whispers, wavering a fiery tail, while vanishing in the air . . . .”

In this piece, the writer frequently applies a type of liner geometry, sometimes parallel, and sometimes crossing. The words “monotonous”, “tail”, “continuously”, “drawing”, “line”, “extended”, “song”, “weaving”, “delusional”, “whispers”, “tail”, and “wavering” all have a linear and continuous geometric component on the inside, which sometimes is interrupted with a vertical geometry, expressed by words such as “heavy”, “soil”, “put”, “crown”, and “head”, which induce a sense of heaviness and burden. Therefore, the writer tries to transfer a sense of entrapment and crucifixion (Hexanoid) at the same time to the readers by drawing numerous intersecting vertical and horizontal lines. This geometry can be extracted, illustrated, and cognitively assessed using the D.P.M. method in a fraction of a second, even from a piece of a novel.

B) The Recognition of a Person's Mental Disorders

Many mental disorders fundamentally impact a person's mathematical mind and hamper the text geometry in certain ways and patterns. These patterns can easily be disclosed using the D.P.M. method, leading to a better diagnosis of psychiatric disorders, with regard to the text math and geometry.

For instance, there are some strong relationships between the type of applied math and geometry by a person and some specific psychiatric disorders. For example, word math can be divided into degradation-decrement math (e.g., analysis, fractional, reduce; which is mostly applied by Cluster B personalities), and incremental-progression math (skyrocketing, increase, prolific). Accordingly, geometry can be classified as dissecting/divisive/dissecting/splitting geometry (e.g., boycott; abstraction; decapitate), or aggregative/unifying geometry (annexation, overlap, repatriation). Typical primitive personalities, such as borderline personalities and those associated with psychopaths, do not have a geometrical mind. They often follow a simple quantitative math or a kind of primitive linear chronology in their own speech. Their penetrative, highly impulsive, and explosive style significantly interferes with any evolutionary and gradual formation of geometrical structures (hexanoids) in their own writing or speech. However, some more well-adapted borderline personality disorders tend to administer a primitive type of linear chronology, or they are inclined to adopt some more specific geometrical patterns on their own speech. Furthermore, they peripherally set some blockades around themselves and then leave a single way out (“one way-out geometry”).

Psychopaths are sometimes seriously interested in depicting a “layer by layer containment geometry”, while penetrating it and ripping it off inwardly. Some obsessive-compulsive patients tend to depict an “equivalent-symmetrical” math; while the others tend to administer linear or parallel geometry more often. Hence, the lack of geometry (as “negative findings”) can present some invaluable information about the person's mentality, as well as its presence and the levels of it.

D13) “Word's 40” Associations Spectrums

Since, according to the D.P.M. method, almost all of the common word's associations have been previously devised and, as of this submission, nearly 20,000 words have been placed on these spectrums, when a spoken or written text is being analyzed using the D.P.M. method, any derailment or loosening of associations would be digitally and automatically recognized and specified, whether its source is mental derangement and creativity, or it originates from lying and fake news. Hence, thousands and thousands of texts could get digitally screened in a fraction of a second. This exclusive property of the D.P.M. method gives it an additionally unique character for “mass detection” of threats and destructive acts, even many years before it happens, therefore improving upon the existing technologies.

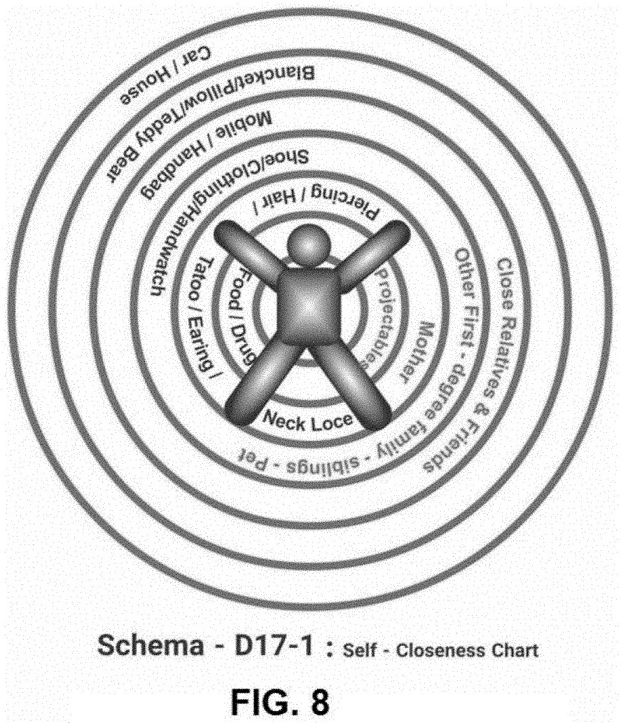

The most renowned word association spectrums include:
1) Colors Spectrum (light colors, junction colors, shadow colors)
2) Size Spectrum
3) Light Reflection Spectrum (shininess, dullness)
4) Penetrative ability Spectrum
5) Penetrability Spectrum
6) Permeability Spectrum
7) Motion Extent Spectrum
8) Speed (Velocity) Spectrum
9) (Potential) Energy Spectrum
10) Height (Altitude) Spectrum
11) Water Content Spectrum
12) Biological Component Spectrum
13) Anti-biology (Harmfulness) property Spectrum
14) Financial Value Spectrum
15) Sex Value Spectrum
16) Edibility Value Spectrum
17) Nesting/Bordering Value Spectrum
18) Word's Math Grade (WMG) Spectrum
19) Words Specificity-Ambiguity Spectrum
20) IMF Spectrum
21) Bordered/Borderless words Spectrum
22) Centripetal/Centrifugal Spectrum
23) Deployment Altitude Spectrum
24) Time-Relatedness Spectrum
25) Self Closeness Spectrum (Da Vinci chart) (as shown in FIG. 8)
26) Severity Spectrum
27) Audibility (Noise) Spectrum
28) Density/Deformability Spectrum

29) Fragility Spectrum

30) Texture Spectrum

31) Emotions Spectrum

32) Existence Possibility Spectrum

33) Positivity-Negativity Spectrum

34) Visibility Spectrum

35) “Awareness-Controllability-Impulsivity” Spectrum

36) “Receptiveness-Action level” Spectrum

37) Predictability-Antochthonous Index

38) Containment Spectrum

39) General Shape Spectrum (Cavity/Flat/Solid)

40) Nominative-Objective Spectrum

As mentioned above, so far nearly 20,000 words have been inserted in these spectrums, and the process still goes on.

D14) Narrow Range Words Vs. Extreme Words

According to the word classification system of the D.P.M. method, there are approximately 40 spectrums of words. In a text, the more the applied words belong to less numbers of these spectrums, the more text would be more specialized (which is indicative of how much a text has been consistently focused on one theme, rather than many different themes and associations). Even within a spectrum, the more the text words are close to each other, consequently the text creator would be more precise, more goal-directed and more cognitively developed (the fundamental fact is that finding differences among very similar things demands more precision and more intelligence).

By contrast, a text that administers a higher number of spectrums and an extensively applied range of words (toward the ends of a spectrum and toward the extreme words), and adopts a more concomitant use of contrast words, would be less specialized and then less intelligently created, respectively.

Relativism (applying more relative words throughout a text) can be considered as a part of the cognitive stability and functionality. Dramatic switches between two ends of a spectrum (The full number of spectrums brought in D-13), which is mentioned in literature as “Contrast”, if it turns to a pattern throughout a person's text or speech, heavily questions his or her mental health. This switching patterns can be seen in OCD patients, as well as borderline personality patients, and ADHD individuals, as well (e.g., “on & off,” “come & go,” “pull & push”). This feature can be easily revealed by “Speech-grams”, as one of the major derivative products of the D.P.M. method.

D15) Word & Text Dysfunction:

In the D.P.M., method, dysfunction has been defined as having one of these characteristics. These characteristics include:

1. Math quantity shortages: the lack of the amount of applied Math & Geometry in a text or speech, or “the poverty of math” in a speech, can be attributed to primitivity, as well as to a lower intelligence.

2. The administration of less (amounts of) geometry (as well as other advanced forms of math): as has been stressed, geometry is the hallmark of highly cognitive function. The lack of adopted geometry in a text can be a sign of lower cognitive ability and lower intelligence.

3. The presence of “degradation-decrement” math, instead of “increment-progression math”: seen in MDD/OCD and cluster B personalities.

4. “Negativity Scales”: (has come in the main table). It is noteworthy that the negativity of a text can be quantified in the D.P.M. method in a chart by the same name (the positivity-negativity chart)

5. Autochthonous/extreme/contrast/impulsive/explosive/bypassing words: the more they are, the less the function would be, because they contradict the continuity, connectivity and the math and geometry, and they also signify an underlying pathology as well as a previous trauma.

6. Increased word and “text fluidity & liquescence”: as seen in classical psychoses like schizophrenia and in severe bipolarity and in pre-psychotic personalities, too.

7. Text Bumpers & Pitholes: they include crossing and d/c words, blockade and barrier words, repetitive words, habitual words and slogans. These type of words and phrases consume and contradict the time and energy significantly, and create a sort of arrest and discontinuity through a speech or text. According to an embodiment, they are adamantly applied by persons to buy enough time to fill their cognitive gaps. Obsessive-compulsive individuals, borderline intellectual functioning people (up to 14 percent of general population who have marginal I.Q.s, as well as chronically psychotic persons tend to adopt these stereotypical types of words and phrases much more frequently.

8. The overuse of ill-defined borders words, quality, ambiguous and over-inclusive words, discussed in D24: as they contradict math and geometry, too.

D16) DPM & Psychiatric Disorders:

Besides some more general aspects of psychiatric disorders like loosening of associations, circumstantiality, flight of ideas, etc., which can be detected by the D.P.M. method, each major psychiatric spectrum (psychoses, mood disorders, OCD spectrum, personality disorders.) has its own distinguishing features in the D.P.M. method. Moreover, some extremely costly neurodegenerative disorders, like Alzheimer's disease, can be digitally and prematurely diagnosed by the D.P.M. method via text messages, tweets, etc., even at very early stages.

To further elaborate, Alzheimer's disease can be easily recognized firstly by a random rating of the ratio of “SOS/whole text words” applied by a patient on his or her mobile or P.C, and throughout a certain period of time, and then by comparing these random ratios with each other, as well as by detecting the lessening of geometric and connectivity values and the addressing capability in their own written or spoken texts.

Bipolarity (Manic phase) can be prematurely detected by rating the person's texts with regard to a dramatically increased libido indices and text kinetic scales, person's action level and outward penetration, while a classical unipolar depression demonstrates a significant decline in the aforementioned scales. Psychosis can also raise the fluidity of membranes; emerging any loss in each of the word's association spectrums can also be also an early sign of psychosis. Furthermore, any rise witnessed in paranoia indices (the main table) in people's written or spoken texts can be prematurely recognized by the D.P.M. method. Therefore, many violent acts and even crimes could be significantly prevented using the D.P.M. method. Personality matters can also be swiftly determined by the D.P.M. method. For example, both antisocial and borderline personality disorders reveal some features in common, including heightened primitivity indices, impulsivity, the presence of bypassing words and phrases, and significantly lowered geometric scales. Nonetheless, borderline personalities show a dramatically raised emotionality index, negativity scales, negatively conflicting words, inwardly penetrating words, and after-burn words, while antisocial personalities reveal much more outward penetration, over positivity, and manipulative words.

OCD Scales

Because of its prevalence, the scales have been brought here in CTp more detail. According to the D.P.M. method, not only can a disorder like OCD be diagnosed through the inspection of a text, but the severity can also be estimated, as well as the resulting dysfunction and the prognosis. The OCD Scales include:

1. The use of Repetitive words

2. Frequently administered “Habitual words”

3. Fulfillment words (e.g., suited; suffice; needed; fully; enough; complete)

4. Symmetry & Equilibrium words (e.g., equally; same; balance; straight; lurching)

5. Frequent usage of “Precision words” (e.g., exact; proper; appropriate; delicate)

6. Frequently used “Minimal size words”: indicative of lower self-esteem (e.g., somehow; a little bit)

7. The presence of “Doubt words” (e.g., depends on; maybe; might; possibly unsure)

8. Oscillatory words (e.g., fluctuation; rebounded; swinging)

9. Raised frequency of Avoidance & Negativity scales

10. Retrospective & Repeated action words (“Re-words”: e.g., repair; reviews; revision; reproach)

11. Repetitive & Habitual words (e.g., frequent use of “You know”, “for example”, “in terms of”, obviously)

12. Frequently applied (simply linear) Comparisons

13. Applying simple linear chronology

14. Negatively interactive words (e.g., challenge, struggling, conflicting)

15. Increased “Verticality”

16. Increased administration of the words that have phonetically conflicting letters combinations

ADHD Scales:

ADHD (in adults and children) is one of the highly prevalent but underreported disorders that impose a heavy toll on a society (because it frequently transforms into the greater community problems like conduct disorder, accidents, and substance dependence). Major diagnostic scales in the D.P.M. method for ADHD include:

1. The overuse of impulsive & explosive & autochthonous & clear-cut words: These type of words are in close contact with “the impatience”, which is dominantly seen in ADHD individuals.

2. The shifting attention scales Including:
2-1) applying oscillatory (“Switch words”): The words that contain an inner element of pendulous, fluctuating, reciprocal movement and act.
2-2) Overuse of contrast words: Another form of switching that probably promotes the process of memorizing in adults with ADHD.
2-3) switching Negativity/Positivity: seen in Negativity-Positivity chart.
2-4) The overuse of the switching prepositions: e.g., but; however; although; against, in contrast.
2-5) Hallmark: (diagnostic) pattern seen in D.P.M. charts (like da Vinci chart).

3P Disorders: (Passivity & Paranoia & PTSD Indices)

3P Disorders essentially refers to three overlapping concepts that all have been shaped around an externally dominant force (analytically named as “the aggressor”). These common areas, however, have their own idiosyncrasies, too. Paranoia, in particular, has its own more specific words, while the other two indices could be recognized through the whole text by detecting the presence of some clusters of words. The indices include:

1. Passivity Indices:

In its purest form, it is a form of learned helplessness and fatalistic thinking and implies the presence of quite less interactive and less dynamic words (e.g., helpless; isolated; alone; frozen; adversity) in a text, along with the presence of concomitant consumptive and disintegrative words; while in a more interactive form it demonstrates a submissive, subdued individual being over controlled, or possessed by a vertically dominant external force (e.g., puppet; robot; over influenced; destined; cursed; spellbound). Passivity can be seen in less pragmatic individuals, and in its mostly extreme form, can be seen in psychotic conditions. The classification has been brought under the title of Passivity scales in the main table.

2. Paranoia Indices:

It generally refers to an extensive spectrum of disorders from some milder forms of paranoia (that can be temperamental or even cultural), to more severe forms, such as paranoid personality disorder or delusional disorder.

Paranoia has its own more specific vocabulary (e.g., victimize; fraudulent; exploiting; injustice; demoralized), although It also has some features in common with other two Ps (Passivity & PTSD). Paranoia reversely correlates with functionality, hence it has been separately brought in the main table.

3. Post-Traumatic-Stress Disorder (PTSD):

PTSD is also a general concept which refers to many disorders that have one major common core; the (history of) trauma, which mostly reveals itself in a text through the presence of after-burn words (e.g.: collapsed; damaged; devastate spoiled), low control (awareness) factors, as well as paradoxical A/P and also other scales referred separately in the main table.

Control Factor

The Control Factor implies to what degree a person feels the things are under his or her own mastery and supervision, which primarily begins with an inner sense of control, or “self-control”, defectively continues with a neurotic over-controlling (of others) and eventually reaches the other end of the spectrum, which is a complete loss of control, or “Impulsivity”.

In the D.P.M. method, almost all of the words (in particular, verbs) have been overviewed in this regard. The lack of a self-control or awareness factor in a text could be demonstrative of formerly traumatic experiences (particularly along with after-burn words), the lower self-esteem, and the lack of confidence about a discussed subject.

Exerting too much control throughout the verbs on other individuals (not the objects) applied by a text creator, or “over-controlling” is also considered to be one of the majorly neurotic defense mechanisms that can be recognized via this index.

Control (Awareness) factors (that has a separate chart and spectrum by this name in the D.P.M. method) can be changed during trauma-related disorders, including acute stress disorder, PTSD, and some more insidiously chronic forms of PTSD, like borderline personality disorder. Therefore, it can be applied as a diagnostic tool for PTSD, too.

Hyperarousal, re-experience, and avoidance have their own distinctive vocabularies that can help the better diagnosis of PTSD; the disorder that is estimated to affect up to 10 percent of general population.

An awareness factor has some degrees, begins with more receptive forms of awareness (e.g., tolerant; endurance), continues with more extroverted forms (e.g., prediction, optimism, planning), and leads to some more action, more exertion of control, and more inductive forms (e.g., directing, managing, suggesting, imposing and other over-controlling roles): adding to this range the zero awareness words, including impulsive and explosive words, the full-spectrum can be reached, in this regard.

Some traumatized personalities, like Borderline Personality Disorders, reveal a switching combination of over-controlling and impulsive-explosive words at the same time in their own spoken or written texts.

D17) Words & Self (Vitruvius or Da Vinci Man)

When a person talks or writes, he or she is actually expanding him or herself and setting their own borders and boundaries in the outer world. To further elaborate, when a person talks, especially when he or she applies some vertically posed objects in their own speech (as language landmarks) as a matter of fact, he or she tries to determine their borders and territories in the outer world.

To clarify the practical implication of this concept, if the scheme is contemplated like the da Vinci man (the “Vitruvius man”) in the center, while surrounded by concentric circles, then we can place almost all the objects on these circles according to their potential closeness to the person's self and body. Scheme D17-1 (FIG. 8) reveals the concept quite well. By administering this chart, we can attain a “bordering & territory chart”.

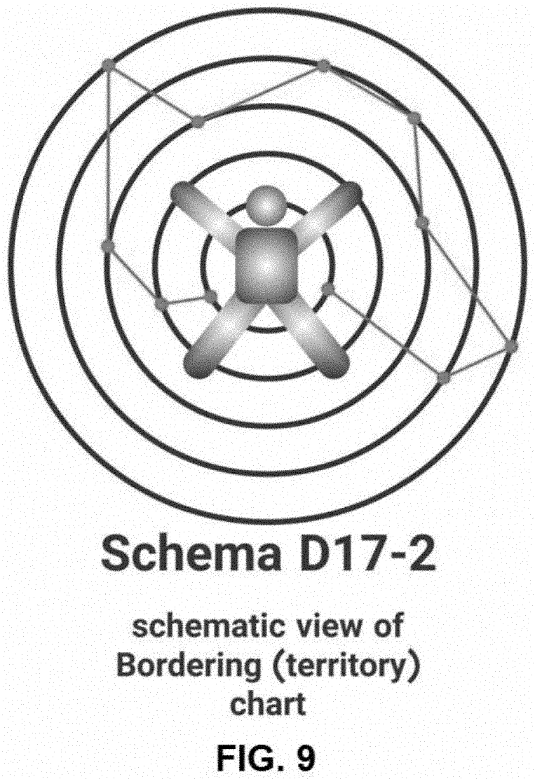

By administering this chart, we can attain a secondarily important chart known as “bordering & territory chart” too. To explain, if we firstly point out the nouns that a person applies during his or her own speech on his or her self-closeness chart and secondly attach those spots by drawing a line (the Schema D17-2 (FIG. 9)), then a chart would be acquired that could be quite specific for each person which is called by the inventor as “Bordering” or “Territory” chart. This chart reveals the person's territory making ability via speech or writing. This chart would be digitally drawn and attained when a person talks by the D.P.M.

By adopting this so-called “da Vinci chart”, firstly some personality issues, like narcissistic personality disorder and also the susceptibility to become home sick (which is renowned as separation anxiety disorder) and also statism (due to OCD, etc.,), can be prematurely diagnosed, predicted, and properly managed; and, secondly, we could have a “bordering chart” for each person which may show some quite characteristic patterns. In other words, the pattern of placing the objects' names and specific nouns around the self can be completely idiosyncratic for each individual. Furthermore, In a more anatomically dissecting look into the person's body parts, some more complementary analytical interpretations can be obtained from the da Vinci chart. To further elaborate, a more detailed category, in terms of body parts, can be attained which contains:

Object, Word & Self Components (Physiologic Map)

1. Relevance to 5 Senses: Sensorium

2. Relevance to major body parts & organs: Shows the way a person connects himself or herself to the external word and the degree he or she is impacting it or is impressed by it). These parts may include hand, foot, lip, abdomen, heart, liver, etc. There are three subtypes:
a. Operant: Movement based organs (head, hands; feet); and contact based organs (Lip, eyes)
b. Expelling parts: More subconscious; mostly applied by impulsive persons; including body outlets like penis, breasts, mouth, anus, womb
c. Receptive/Susceptible/Submissive parts: Pertinent more to integrity anxiety, e.g., ears, liver, bowels, anus, mouth

D18) Other Spatial Deployment Charts

Objects can also be classified according to their other deployment patterns in the space, including the layer by layer containment property. To further elaborate, many objects can be sorted based on this property in which one is placed within the other.

Containment associations become mostly important when we are going to recognize penetration (inwardly or outwardly), which is mostly seen in cluster B traits and personalities. Besides, vertically deployed objects and movements, herein referred to as “Verticality”, can be characteristic of obsessive-compulsive disorder. There are much more spatial deployment patterns that each one implies a particular trait of personality or a mental state. These patterns can also be categorized in the text geometry by the D.P.M. method, too.

D19) Psychic Map

If the basic and simple schema can be regarded as revealed in the schema-D19. Then, many words can be psychologically classified in a text, as follows:

1) Self representatives (projectable things)

2) Maternal figures

3) Paternal-authority figures

4) Libidinal figures (male type/female type)

5) Transitional objects

6) Rivalry figures

7) Aggressor figures

8) Transcendental figures

9) Mating/Fertility figures

10) Nesting & Territory-making figures

11) Hunting figures (& Esteem roles & Dominance roles)

Each of these subtitles has its own vocabulary in the D.P.M. method. Hence, this schema simply demonstrates that the D.P.M. method has the capability to go subjectively deeper and deeper, in order to explore even some of the more psychoanalytic aspects of the human mind.

The D.P.M. method can artistically, objectively, and extraordinarily rapidly interpret texts and even extract psychoanalytical clues from it within a second. For example, the D.P.M. method swiftly perceives a separation anxiety as well as an obsession from this sentence: “She felt cold deeply inside his feet, but the woolen sucks gave him more strength to walk.” “Deeply” and “inside” are static words on the first part of the sentence (which has been negatively expressed); while the second part has more dynamism, using words like “feet” and “walk”. So a sense of hesitation between the two contrasting poles in the sentence can be observed. “Sucks” represents a sense of attachment, along with feeling “cold”, which is symbolically indicative of separation anxiety. The whole sentence lacks the math and geometry, too (Negative findings). As a result, the writer of the sentence who is probably an emotional and tactile person, tries to express the sense of hesitation to leave (sense of rootedness), and attachment, which is mostly demonstrative of separation anxiety, but he is finally positive, as he has ended the sentence more dynamically and more positively. Therefore, the writer likely suffers from OCD and SAD and is also primitive in thinking (making contrast but lacks mathematics throughout his or her sentences). He is positive at the end, thus the severity of his or her symptoms are likely low.

On the next levels, each of these preliminary clinical and analytical judgments can be established and are also more accurately rated in terms of severity and fiction by hearing further sentences from the sentence writer. According to an embodiment, the D.P.M. method enables a user to disclose defense mechanisms in a text, too. Reaction formation and denial, nonetheless, can be at the moment revealed by a negativity-positivity chart, as well as over-controlling that has been discussed earlier.

D20) Positivity-Negativity Chart

Switching from positivity to negativity can not only be seen in ADHD, but also can be detected in OCD patients who frequently have doubtful thoughts. Patients who have tic disorder may also reveal the similar pattern (oscillating between positivity and negativity, with the dominance of negativity) in their own speech. Therefore, these three commonly relevant psychiatric disorders can also be diagnosed from people's speech, by recognizing their switching patterns on the positivity-negativity chart.

This chart also can reveal the range of emotions, as well as the neurotic features of a person. Personality disorders also reveal certain diagnostic patterns in this chart. For instance, over positivity can be expressed by liars and psychopaths, while over negativity is mostly revealed by paranoid and borderline personalities (negativity scales have been completely defined in the D.P.M. method, brought in the main table).

D21) DPM & Sentence Assessment

As repeatedly mentioned before, the D.P.M. method not only analyzes words, one by one, but also has an enormous capability to analyze whole sentences of a text.

From an evolutionary point of view, probably the most ancient and primary grammatical components are nouns and names. They collectively constitute the speech or text skeleton. Adjectives are other grammatical elements that would mainly attach to nouns and names, afterwards. Verbs are considered to be historically newer grammatical elements of a sentence, and adverbs, which define and clarify the verbs (mostly), are probably the newest evolutionary elements of any text or speech. Hence, generally speaking, it can be deduced that primitive speech likely lacks more advanced elements of speech, such as adverbs, as well as a lesser range of verbs. In contrast, more specialized and more mature speech contains a smaller range of names and adjectives, and a greater applied range of verbs, along with their attaching elements, which are mainly the adverbs.

Sentence assessment has other important aspects, too. The distance among grammatical elements in a sentence differs significantly from person to person. For example, the significantly lowered distances between subject and object in a text can be a sign of impulsivity, which can be dominantly observed in individuals with cluster B personality.

Additionally, the length of the applied sentences could vary among people. However, frequently applied highly shortened sentences can be seen in individuals with borderline personalities and in psychopaths, who essentially lack the development of more advanced linguistic structures. On the contrary, the presence of repetitively over-lengthened sentences, insisting on applying all the grammatical elements together, particularly around one theme, can be the sign of OCD.

The simplicity or complexity of applied sentences can also have other aspects. For instance, the presence of combinational sentences with a high frequency of applied gerunds, infinitives, and adverbs can be a sign of increased abstract thinking, as well as obsessive-compulsive personality disorder (OCPD). Hence, the D.P.M. method digitally and exquisitely analyses the sentences as well as the words and the whole text.

D22) Word Memorability

Word memorability refers to the degree that a person can remember words. Memorable words act like landmarks that for their own sake an individual can remember the whole text much rather than for the sake of gerunds, verbs, action nouns, etc. This property can become hampered by some neurodegenerative disorders such as Alzheimer's, or even in ADHD and severe OCD.

According to word memorability some of the mostly memorable words as can be ranked as:

1. Specific nouns

2. Specific words like: vertical object names (landmarks)/places names/SOS-contained words

3. Extreme words & Contrasting words

4. Pop-up words(words that are phonetically or semantically bolder and more distinct than the adjacent words.

D23) Dissecting Literature (Deformity & D/C Words)

On the opposite verge of important concepts such as “Connectivity”, “continuity”, and being constructive, there are some kinds of negatively connoted, and often high action level words and literature that are nearly as important as the formers, because convey us to more diagnostic clues. This type of words are more interruptive, disruptive, disconnecting and destructive, and are considered as a part of primitive, immature, or trauma-induced thinking. The latter part (trauma) has a great role in the evolution of this type of literature in a person, since many clusters of these words reenact a facet of a multifaceted trauma that probably had been painfully experienced by the person in the past. For example, autochthonous words that have suddenly emerged and come out of the blue (e.g., all of sudden; popping out; accidental; erupted) may reveal a sudden capturing feature of a previous trauma in the person's anamnesis or a person who may apply too much after-burn words throughout speech or text, probably is revealing the overwhelmingly destructive aspect of a previous trauma, reenacted again via his or her current speech or text. Metaphorically speaking, these types of words belong to the very ancient times rather than the modern world, and to the darker and destructive side of the world, rather than the brighter and constructive side. Therefore, primitive thinkers, such as adults with ADHD as well as immature personalities (including individuals with borderline personalities and psychopaths) are much more interested in applying these type of words and literature because of their split self, their lack of enough patience to achieve the goals, a continuous history of failures and under achievements, and also their inability to understand the cause and effect relationships and their more linearly straight forward, more penetrative, and destructive approach toward life, rather than the a gradual and step by step construction, which obviously demands patience and dedication. Main clusters of this literature include:

Impulsive Words:

Impulsive words reveal a sudden act or happening that occurs during a short amount of time (e.g., hurry up; quickly; rushed into; kick oft). They exhibit a sense of rush, impatience, and jumping into final conclusions via shortcuts. They are quite similar to explosive words, but are presently separated, since the latter might reveal more destruction in essence and more negative connotation, as well.

Explosive Words:

Explosive words often refer to a sudden burst of things, or the presence of a bursting capability within objects, used more often by individuals with intermittently explosive personalities. Explosive words have an inner destructive or consumptive element, too (e.g., blew up; crashed; clashing with).

Autochthonous Words:

The psychiatric meaning of autochthonous words differs from the popular one; it often refers to a suddenly emerging and coming out of the blue (or, in contrast, suddenly disappearing) act or event during a speech or text. These words come to reality or disappear unexpectedly and with no relevant cause and effect or no prognostic signs; these type of words often have a tight connection with visual senses (e.g., occurred; appeared; surprised; loomed; popped-up).

Clear-Cut Words:

Refers to a type of words that puts an end to an act or draws a line or border within a word or amongst the words. It has an inner start-point and endpoint, splitting and cutting propensity (e.g., blockade; ruled out; cut-off). Clear-cut words mainly have clear-cut antonyms, too (e.g., terminable vs. interminable; catchable vs. uncatchable). Clear-cut words are often used by persons with higher action levels, and higher degrees of pragmatism as well.

Bypassing Words:

Bypassing words can be considered as a subtype of impulsive words and refer to a shortcut pathway in terms of meaning (ditched; skipped; hijacked) or in terms of form(e.g., STMU; Obvi).

Extreme Words:

Extreme words generally refer to words that have been placed onto the endings of each of the 40 spectrums. Hence, not only “hottest”, “least”, and “finest” are considered to be extreme words, but also words such as “steel”. “toxins”, “sunburn”, and “virus” could be regarded as extreme words, since they have been settled on the ends of density, vitality, temperature, and size spectrums, respectively.

Contrasting Words:

Words that have been placed on the two extreme sides of each association spectrum can be contrasting toward each other. e.g., dusk and dawn. These are contrasting words. In much more subtle and insidious forms, and in central portions of each spectrum, words can mildly contrast each other (e.g., toxins vs. drugs). Revealing all of these mildly contradictory words is only possible by applying a digital system like the D.P.M. method.

What is the importance of disclosing the contrasting words in a text or speech? The answer is that the presence of deeper and more severe contrasts strongly correlates with the presence of primitivity as well as internal conflicts and psychopathology.

On the opposite side, there are “transforming words” that not only have an inner time element but also contain a gradation element on the inside. They often refer to a gradual and more often a continuous process (and sometimes a step-by-step process) rather than a sudden start or closure, cut-offs, or a quick and unexpectedly impulsive act. (e.g., wear off; secondly; lingering; prioritizing; prolonging; carry on). They are often applied by tolerant and mature individuals (in contrast with quality words that are clear cut words overused by primitive personalities).

It is noteworthy to know that not all of the deformity and D/C words have the same values. A range of functionality even among D/C and Deformity words can therefore be conceived. Some forms have less severity than the others. For instance, “being negatively impacted” has a less destructive degree than “being damaged”, and the latter one owns lesser severity than “being devastated”, in this regard. Besides, some forms of math and geometry can be recognized even within the D/C words. For example words such as “denuclearization” or “decapitate” have an internal within solid (three-dimensional) geometry while words such as “deforestation” and “desertification” have more plane (flat) geometry. Furthermore, words such as “derailed” or “dislocated” have an inner linear geometry, and words such as “biodegradable” and “de-energized” have more quantitative values than geometric values. Therefore, even deformity and destructive words can be ranked according to their severity, as well as their internal type of applied math and geometry.

D24) Physically (or Semantically) Ill-Defined Words

Physically (or semantically) ill-defined words include three majorly overlapping clusters. These clusters include:

1) Borderless Words (Quality Words)

Borderless words often include popular and abstract nouns and generally refer to the types of words that do not have any distinct borders, serious geometry, or even highly distinct localization. “Unethical”, “humanitarian”, “artistic”, “evaporation”, “detergents”, and “fair” are examples of words with roots for whom a distinct border, geometry, or localization cannot be perceived. Quality words, along with ambiguity and over-inclusive words, make a triangle of non-mathematical and borderless words.

2) Over-Inclusive Words (Word Inclusiveness or Self-Contained Words)

Over-inclusive words are considered to be a subtype of borderless words. Over-inclusiveness refers to the collective presence of too many elements (interim SOSs, the distinct atmosphere, inner geometry, etc.,) within an abstract noun without a within integrating geometry; the property that not only excludes the word from the specificity zone, but also gives it a more general, and at the same time more meaningfully vague, global, and insufficient aspect.

This type of word appears to be semantically independent from the other words (e.g., biology; unethical; humanity; benevolence; consensus; artistic; cosmic). Over-inclusive words can be seen in any text, but the overuse of them, which is essentially more static, less directed toward the other words, and much less serving the whole meaning of the text, could be seen in educated OCD individuals, in individuals with OCPD personalities, and in some types of psychoses. In general, these words are used by people who are overly preoccupied with comprehensive concepts and with his or her own inner world.

As previously mentioned, word inclusiveness is remarkably different from word specificity; i.e., “investigation” and “research” are inclusive words, whereas words such as “perspiration”, “over capitalism”, and “monetizing” are considered to be specific words.

3) Ambiguity Words & Ambiguity Scales

Ambiguity of a text implies a text insufficiency in terms of communicating and reaching a point and a meaning directly and explicitly by a reader or listener. It generally occurs by reason of administering general and vague words (e.g., anybody, something, people, happiness, prosperity, humanity, whoever, etc.) or choosing wrong or loose associations.

Ambiguity scales reveal how much a person is keen to (or knows about) a matter, an object, or a person. By combining the ambiguity chart with the da Vinci chart, valuable information can be acquired about a person's knowledge and the real trend towards the discussed subjects, because, when people do not know, or are quite reluctant about a matter, an object, or a third person, the person's range of applied words tend to become more vague and more ambiguous. The ambiguity scales include:

1. the overuse of gerunds and action nouns

2. the overuse of common nouns

3. the overuse of abstract nouns

4. the use of over-inclusive words

D25) Digital Psychometry & Fake News

According to an embodiment, the D.P.M. method does not have such a claim that could promptly address the distorted or fake news (since some of those are too delicately designed to be recognized without more exploration). It, however can have some estimates about their whereabouts within a text, and the possibility of their happening. To explain, considering the libido charts (discussed in D2), text rhythm chart(suggested in D11) and the discrepancy between “the expected math” (Schema D25) and the existing math “within a text,” then any significant change or contrast in terms of these scales will raise the possibility of ongoing inconsistencies including conflicts, lying possibility and fake news. It becomes much more important in the process of “mass screening” of texts with this regard.

Some examples of“the expected math” (any discrepancy witnessed among the expected math and the existent one could be a sign of malfunctioning, inconsistencies as well as distorted and fake news) includes:
IDEAL KINETICS owns:
   distinct angle and direction
   distinct velocity & acceleration
   distinct movement pattern
IDEAL CONNECTIVITY contains:
   lowest destruction
   less velocity
   higher accuracy
   higher rigidity
IDEAL DETACHMENT (always less functional) has:
   less velocity
   less fluidity
   less emotions around
   less destruction

D26) Digital Psychometry & the Overall Text Value

According to an embodiment, if concrete words, (quantitative) math words and geometric words on the one side and ill-defined words (brought in detail in D24) on the other, be regarded as the major function and communication role players in a written or spoken text, then we could have an overall estimation about a text (functional) value with regarding the proportions of these types of words toward each other. Therefore for instance, the ratio of geometric words to ill-defined words (like over-inclusive words who do not have any distinct borders or time or place relevance) can not only disclose a text cognitive and scientific value, but also can reveal its overall functional value in a glance. This view becomes more important in the “digital mass screenings” of many written or spoken texts, whether they holistically assess and/or carry more valuable information in terms of their functional and communicative values or not.

D27) Digital Psychometry & Gender Related Matters

If the fact that femininity and masculinity have some revelations in human's speech is contemplated, then the degree of manhood and also feminism throughout people's spoken or written texts can be reasonably estimated. To further explain, phallic figures (particularly erected, solid, and vertical ones) can “digitally” be recognized within a text as well as the cavernous, hollowed and hole-like figures (representative of feminism). Moreover, the choice of verbs (infinitives and gerunds) could demonstrate to what degree a person is compatible, submissive, and/or receptive (e.g., as femininity features in men), or challenging, oppositional and bullish (e.g., as manhood characteristics in some females).

According to an embodiment, the whole text summation, with this regard, could reveal the degree of feminism or manhood in men and women respectively. In terms of this, we can have a “Protrusion—Depression Chart” (herein also referred to as a Hole-Bar chart) which can be digitally charted for each text and can reveal some more aspects of gender-related idiosyncrasies of the text owner.

What's the importance of this? It mainly goes back to the type of character and the choices of jobs that people have. Picking a man with a high degree of femininity for mining, combat, and artillery jobs then would be probably less functional as selecting a woman with her choice of manhood words for a fine embroidery job. Besides, the presence of the holes, hollowed and cavernous spaces on the one side, and solid, protruded and erected objects on the other within a text, can be in connection with the inner senses of emptiness, after-burn feelings, and previous traumatic experiences or, in contrast, can be attributed to the presence of the integrity, determination, and higher self-esteem, respectively. Hence, a hole-bar chart can help increase recognition of these mental states, as well.

To sum up, the D.P.M. method can swiftly, objectively, and digitally estimate the psychosexual idiosyncrasies according to the people's choices of words (picking hollowed ones or solid ones), and, therefore, an employer can hire a more fitted person from long distance with more certainty, regarding these features, and an insurer can either have a more reasonable hunch about his or her decisions with this regard.

This has to be seen as an objective and realistic perspective towards both men and women who carry another's sex characteristics within themselves, rather than a discriminating gender perspective.

D28) D.P.M. & Self Steem

According to an embodiment, the combination of three associations, including “the size related associations”, “the verticality associations”, and “the self-closeness chart” in the D.P.M. method can digitally determine the degree of genuine self-confidence and self-esteem that an individual reveals through his or her own speech or text. This can be digitally assessed and demonstrated by the D.P.M. method.

### The Software Component

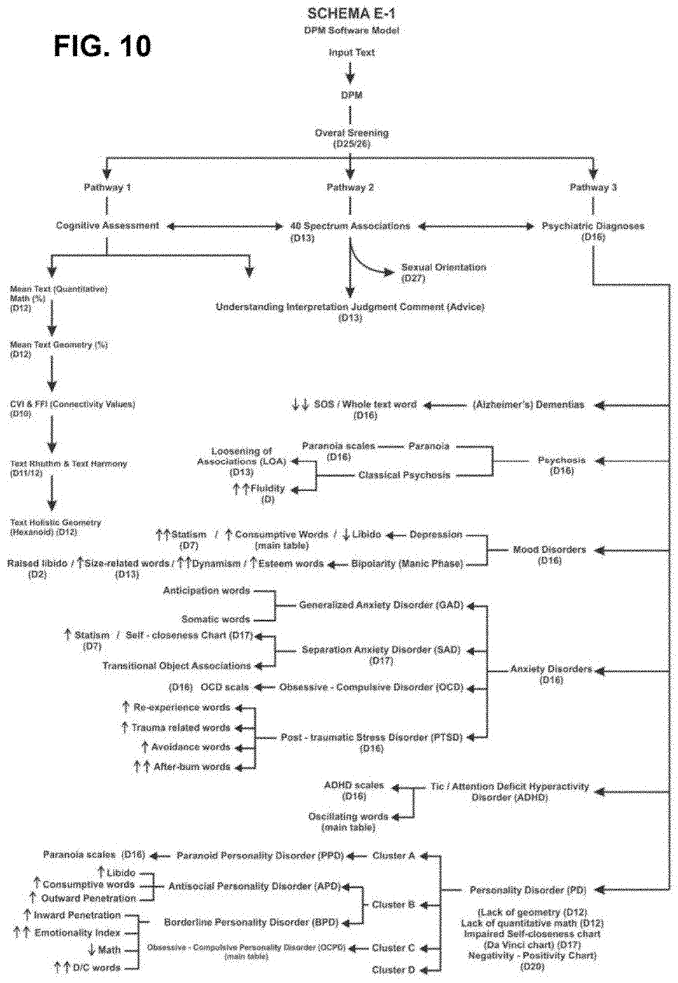

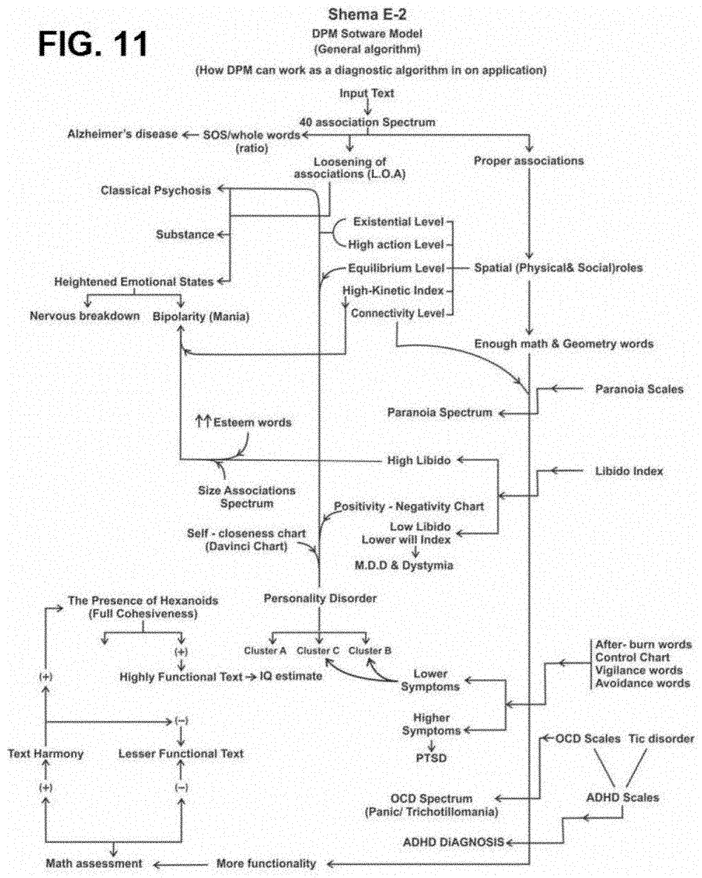

The diagnostic global algorithms proposed (The schemes E-1 and E-2, as shown in FIGS. 10 and 11, respectfully) easily reveal how a software can be devised according to the major principles of the D.P.M. method and ensue in a better digitally cognitive and psychological recognition of a written or spoken text, as well as its own creator's mind.

The schemes E-1 and E-2 are practical flow charts that are representative of how all the theoretical principles of D1-D27 could be exploited in a diagnostic algorithm as the foundation of a DPM-based application and software.

The following global algorithm reveals how the computer-based main part of the D.P.M. method can result in a cognitive and psychological recognition of a written or spoken text, as well as its own creator's mind:

Practical Implications of the D.P.M. Method:

The D.P.M. method can be regarded as the major missing part of Artificial Intelligence (A.I.). To further explain, in recent years, A.I. has dramatically progressed in simulating body movements (by inventing humanoid robots), and many successful efforts have been also made in the domain of sensations (by creating devices such as virtual reality headsets and sex-bots), calculations, and predictions (chess-playing robots and calculators), and memory (as can be seen in the extraordinary capability of restoring huge quantities of information with the ability of instant recalling). However, in the domain of thought and understanding, very little success has been made so far. Therefore, the D.P.M. method can be considered as the missing part, since it can analyze, interpret, get the clue (meaning) and then appropriately respond (logically or emphatically). Because the D.P.M. method can disclose and properly touch on all of the words' associations (40 spectrums in D13) and can conform them with a neuro-cognitive and psychic map, it can “interpret” and “understand” the speech elements in a comprehensive manner that no human being can achieve. Hence, the D.P.M. method is assumed to be a game changer in this domain and an improvement upon the existing technology.

According to an embodiment, the D.P.M. method is incorporated into an electronic device. According to an embodiment, the electronic device may be a desktop computer, a laptop computer, a tablet computer, a smart phone, and/or any other suitable electronic device. According to an embodiment, the D.P.M. method is run on an application. The application may be used to overcome people's judgments and denials and incomparably raise the understanding of human beings to a higher level; and beyond that, it could profoundly empathize and care for other's feelings and emotions; an ability that current companion robots lack.

Furthermore, the D.P.M. method has an enormous capability of “mass screening” of (cognitively) highly functional people, as well as risk-taking ones, such as extremists, terrorists, criminals, and liars, according to their speech/texts in cyberspace. These computations can be completed within a fraction of a second; even when they are not talking directly about something serious, because the D.P.M. method more specifically focuses on an individual's “thought process” rather than the individual's thought content, the element that cannot be easily concealed or forged by people. It can also be a substantially reliable diagnostic substitute for some former psychological and psychiatric “diagnostic” systems such as DSM and ICD, which are defectively more reliant on possible behaviors, and it can provide a higher sensitivity, a desirable specificity, and a swift diagnostic approach toward people's minds in order to detect their psychiatric afflictions, such as depression, OCD, and bipolarity. It is noted that the D.P.M. method's judgment is not based upon some catastrophically confounding factors in the field of diagnosis and treatment follow-ups, like attitudes and feelings of the therapist, as well as false counter-transferences. It can also diagnose some major neurological disorders, such as Alzheimer's disease, at quite early stages and consequently would dramatically lessen their huge burden posed to healthcare systems and insurance industries. The D.P.M. method, moreover, can give a prognosis about the stability of people's relationships and their marriages when they are even at the age of 5, by defining a “Separation Index” as a part of the psychic map. Even separation anxiety and future susceptibility of homesickness can be previously detected and prevented by the D.P.M. method. Besides these, munch more advanced and specialized dictionaries can be designed and provided based upon the methodology of the D.P.M. method, which is a multidimensional analysis of a word, as well as revealing all its essential associations.

The D.P.M. method also can be the main constituent part of “speech-grams”; the instruments that could change people's speech to interpretable figures and charts. Finally, many subjective fields can be fundamentally impacted by the D.P.M. method, as it can provide them a unitary, objective, and evidence-based perspective in fields such as literary criticism, literature, linguistics, neuropsychology, clinical psychology and psychiatry.

The D.P.M. Method's Illustrating Capability:

The D.P.M. method has an enormous capability to turn speech into objective and attractive charts and graphs. For example, it not only can quantify speech ingredients and put them into certain diagnostic categories, but also can turn speech into objective and referable graphs and charts. As a result, psychologists and psychiatrists come to conclusions by taking a quick look at these graphs and charts. Judges can also objectively see the degree of offense, wrongdoing, or affliction, and, most importantly, people, themselves, can overcome their fears and denials about being psychologically naked in front of a stranger and instead can evidently and simply watch and explore what is wrong about them, their children or their distant relationships

Some importantly suggested charts and graphs that can be created using the D.P.M. method include:

Speech-Grams:

Speech-grams resemble an EEG. To clarify, as a patient/person talks, the speech gram (which has been built according to the D.P.M. software) can draw a linear graph on a computer screen or on a paper, revealing the speech continuity, circumstantiality, derailment, flight of ideas, lapses and pauses, preservations, as well as many other qualities. These could be attached to a patient's file and inspected by an expert. These can be an objective basis for follow-ups and changing treatment plans. They can also resolve diagnostic arguments among experts which often stem from personal attitudes that mostly damage the patients during treatments.

Da Vinci Chart (and Bordering or Territory Chart):

Da Vinci charts can reveal how a person makes their own territory beyond himself or herself by choosing objects' names and nouns. Reviewing the chart will give a snapshot of how a person's linguistic and cognitive territory is spread out beyond his or her personal conflicts.

Negativity-Positivity Charts:

Negativity-Positivity charts majorly reveal people's neurotic qualities, as well as the severity of their defective defense mechanisms applied throughout their speech.

Libido & Geometry Charts:

These charts represent “continuum concepts” of libido, connectivity, math, and geometry, respectively. In the most primitive form, people's libido can be shown in the shape of colored charts, conveying the observer to the degree of primitivity, desire, and vitality ups and downs. These charts can be quite helpful in the swift diagnosis of MDD, bipolarity, and Alzheimer's, too.

Interestingly, people are heavily interested in knowing how much chemistry and desire has been expressed through a text by their own lover, as well. On the next level, and as the precursor of geometry, continuity/connectivity charts demonstrate to what degree a person tries to create a properly aligned, continuous, cohesive, connective, and communicative speech or text by the right choice of (relevant) words. Conversely, these charts also can reveal the degree of disconnecting property, misalignments, chaos, and deformities throughout the spoken or written texts and, in mostly advanced form, “the geometry chart” tries to represent the summed-up word angles and directions as well as “the text rhythm” and “harmony” and also tries to extract and reveal the possibly existent geometric shapes in order to reveal the text owner's intelligence and cognitive ability.

Awareness & Control Charts:

Awareness and control charts reveal to what degree a person is conscious about what he or she says or writes. Awareness words are often receptive words with a concomitant sense of insight and thoughtfulness. Over-controlling words carry lesser amounts of insight and consciousness, but higher amounts of action level Clear-cut, impulsive, explosive words that are considered to be quite subconscious, can also be placed on the extreme side of awareness and control charts.

Hence, the illustrative ability of the D.P.M. method, along with its quantifiability, makes it more objective, more evidence-based, and more charming for people.

When introducing elements of the present disclosure or the embodiment(s) thereof, the articles “a.” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method for analyzing one or more psychological states of a user, comprising:
analyzing, using a first electronic computing device, a plurality of words, wherein the second electronic computing device includes a processor and a memory;
organizing, using the processor of the first electronic device, each word in the plurality of words into one or more categories based on the analysis of the plurality of words;
receiving a plurality of words from the user;
analyzing, using a second electronic computing device, the plurality of words from the user, wherein the second electronic computing device includes a processor and a memory;
determining, using the processor of the second electronic computing device, which category or categories to which one or more words in the plurality of words from the user belongs;
determining, based on the analysis of the plurality of words from the user and the determination of which category or categories to which the one or more words in the plurality of words from the user belongs, one or more psychological states of the user; and
outputting, to a graphical user interface, the one or more psychological states.

2. The method as recited in claim 1, wherein the plurality of words are derived from written or spoken text.

3. The method as recited in claim 1, further comprising:
rating the one or more words in the plurality of words from the user within the determined category or categories.

4. The method as recited in claim 1, wherein the one or more psychological states are selected from the group consisting of Intelligence Quotient (“I.Q.”); mood; thought content; thought process; and personality traits.

5. The method as recited in claim 1, further comprising:
preparing, using the one or more determined psychological states, an assessment of any possible psychological conditions the user may have.

6. The method as recited in claim 5, wherein the psychological conditions are selected from the group consisting of: major depressive disorder; neuro-degenerative disorders; Alzheimer's disease; obsessive compulsive disorder; bipolarity, separation anxiety disorder; attention deficit hyperactivity disorder; borderline personality disorder; and psychosis.

7. The method as recited in claim 1, further comprising:
analyzing at least one string of two or more of the plurality of words from the user.

8. The method as recited in claim 7, further comprising:
determining which category or categories to which the at least one string belongs.

9. The method as recited in claim 8, further comprising:
determining, based on the analysis of the at least one string and the determination of which category or categories to which the at least one string belongs, one or more psychological states of the user.

10. The method as recited in claim 1, wherein the second electronic computing device is selected from the group consisting of a desktop computer; a laptop computer; a smart phone;
and a tablet computer.

11. A system for analyzing one or more psychological states of a user, comprising:
a first electronic computing device, including:
a processor configure to:
analyze a plurality of words; and
organize each word in the plurality of words into one or more categories based on the analysis of the plurality of words; and
a memory configured to store the analyzed plurality of words; and
a second electronic computing device, including:
a memory;
a graphical user interface configured to receive a plurality of words from the user; and
a processor configured to:
analyze the plurality of words from the user;
determine which category or categories to which one or more words in the plurality of words from the user belongs;
determine, based on the analysis of the plurality of words from the user and the determination of which category or categories to which the one or more words in the plurality of words from the user belongs, one or more psychological states of the user; and
output, to the graphical user interface, the one or more psychological states.

12. The system as recited in claim 11, wherein the plurality of words are derived from written or spoken text.

13. The system as recited in claim 11, wherein the processor of the first electronic computing device is further configured to:
rate the one or more words in the plurality of words from the user within the determined category or categories.

14. The system as recited in claim 11, wherein the one or more psychological states are selected from the group consisting of Intelligence Quotient (“I.Q.”); mood; thought content; thought process; and personality traits.

15. The system as recited in claim 11, wherein the processor of the second electronic computing device is further configured to:
prepare, using the one or more determined psychological states, an assessment of any possible psychological conditions the user may have.

16. The system as recited in claim 15, wherein the psychological conditions are selected from the group consisting of: major depressive disorder; neuro-degenerative disorders; Alzheimer's disease; obsessive compulsive disorder; bipolarity, separation anxiety disorder; attention deficit hyperactivity disorder; borderline personality disorder; and psychosis.

17. The system as recited in claim 11, wherein the processor of the first electronic computing device is further configured to:
analyze at least one string of two or more of the plurality of words from the user.

18. The system as recited in claim 17, wherein the processor of the first electronic computing device is further configured to:
determine which category or categories to which the at least one string belongs.

19. The system as recited in claim 18, wherein the processor of the first electronic computing device is further configured to:
determine, based on the analysis of the at least one string and the determination of which category or categories to which the at least one string belongs, one or more psychological states of the user.

20. The system as recited in claim 11, wherein the first electronic computing device is selected from the group consisting of a desktop computer; a laptop computer; a smart phone; and a tablet computer.
